(19) United States
(12) Patent Application Publication
(10) Pub. No.: US 2021/0077673 A1
(43) Pub. Date: Mar. 18, 2021

(54) Container Assemblies For A Breast Pump

(71) Applicant: Willow Innovations, Inc., Mountain View, CA (US)

(72) Inventors: Brian Mason, Lexington, MA (US); Jenny Teng, Belmont, CA (US); Taylor Walsh, Philadelphia, PA (US); Erica Keenan, San Francisco, CA (US); Fred Co, Santa Clara, CA (US); John Chang, Los Altos, CA (US)

(21) Appl. No.: 16/951,202

(22) Filed: Nov. 18, 2020

Related U.S. Application Data
(63) Continuation of application No. PCT/US19/33033, filed on May 20, 2019.
(60) Provisional application No. 62/788,794, filed on Jan. 5, 2019.
(60) Provisional application No. 62/776,565, filed on Dec. 7, 2018.
(60) Provisional application No. 62/674,545, filed on May 21, 2018.

(51) Int. Cl. A61M 1/06; A61M 1/00

(52) U.S. Cl. CPC A61M 1/062; A61M 1/0005

(57) ABSTRACT

Systems and methods for pumping milk from a breast, wherein the milk is expressed from the breast under suction and milk is expulsed from the pumping mechanism to a collection container assembly.

### FIELD OF THE DISCLOSURE

The present disclosure generally relates to container assemblies for portable breast pump systems and methods for collecting milk from a breast of a nursing mother.

### BACKGROUND OF THE DISCLOSURE

As more women become aware that breastfeeding is the best source of nutrition for a baby, and also offers health benefits to the nursing mother, the need is increasing for breast pump solutions that are user-friendly, quiet, discrete and versatile for use by a nursing mother in various situations. This is particularly true for the working mother, who is away from the home for eight to ten hours or more and needs to pump breast milk in order to have it available for her baby, but it is also a requirement for many other situations where the mother is away from the privacy of the home for an extended period, such as during shopping, going out to dinner or other activities.

Although a variety of breast pumps are available, a number are awkward and cumbersome, requiring many parts and assemblies and being difficult to transport. Hand pump varieties that are manually driven are onerous to use and can be inconvenient to use. Some powered breast pumps require an AC power source to plug into during use. Some systems are battery driven, but draw down the battery power fairly rapidly as the motorized pump continuously operates to maintain suction during the milk extraction process. Certain other of the breast pumps available are lacking in convenient storage containers. The storage containers can be difficult to package, install, remove and store thereby creating a barrier to effective use.

There is thus a continuing need for conveniently usable and effective container assemblies for portable wearable breast pump. The present disclosure addresses these and other needs.

### SUMMARY OF THE DISCLOSURE

Briefly and in general terms, the present disclosure is directed toward container assemblies for a breast pump system. The system includes breast contacting structure and a storage container, and structure that delivers milk or other liquid from a breast to the storage container. The method involves pumping milk from a breast and delivering the pumped milk into the container assembly. Various of the disclosed container assemblies are disposable while others are reusable or include portions that are disposable or reusable, and various portions of the container assemblies are made from one or more of rigid or flexible materials.

According to one aspect of the present disclosure, a system for pumping milk from a breast includes a container assembly in combination with one or more of: a skin contact member or flange configured to form a seal with the breast; a conduit in fluid communication with and connected to the skin contact member; a driving mechanism configured to establish a vacuum profile within the conduit; an external shell; and a non-transitory computer readable medium having stored thereon instructions executable by a computing device to cause the computing devices to perform functions associated with and directed by the firmware; wherein the external shell comprises a compartment facing a distal end of the external shell, the external shell further comprising a proximal end surface facing away from the proximal end; wherein the skin contact member, the conduit and the driving mechanism are received in the compartment of the external shell; wherein the milk collection container is positionable within the shell; and wherein the system is shaped and configured to be contoured to the breast of a user.

In various embodiments, the storage container includes a body and a spout extending from the body. The body can further include a central opening generally defining an oval shape or can include one or more slits in the central portion of the body of the container. In additional or alternative embodiments, a second spout extends from the body, and the body includes a vent for removing air. Various internal seals and patterns thereof are formed in the interior of the body in alternative or additional approaches to direct fluid and minimize leaking. In one or more embodiments, dual film valve structure is configured within the container body, the structure also configured to one or more of direct fluid and minimize leaking. Various approaches and structure of fitments are attached to the various embodiments of the container assembly body such as dual boss, folding, plug, ring, clips and inverted boss structure is arranged to create a seal as well as structure that mates with corresponding breast pump system assemblies. In one aspect, fitment to cap interfaces are unique and non-round. In other related structure, there is provided a fluid container to fluid container connector.

In other approaches, the container assembly is formed from reusable materials and structure which can be either rigid or flexible or includes portions that are rigid or flexible. The container assembly can be composed of one piece or multiple pieces. In one embodiment, the container assembly is longitudinally split and includes a first part connected to a second part via welding or a seal along a perimeter of the two parts, and further includes a funnel defining a nipple receiving portion that completes the assembly. The assembly can alternatively or additionally include a removeable plug facilitating access to an interior of the container assembly or for removing collected liquids. In another embodiment, the container assembly is split laterally and includes a first part attached or attachable to a second part and further includes a funnel defining a nipple receiving portion that completes the assembly. Various other dividing and attachment edges between subcomponent container assembly parts are also provided in certain embodiments, and the container assembly can define a cavity integral with flange structure. The assembly can alternatively or additionally include a removeable cap that provides access to an interior of the container. In additional or alternative aspects, the container assembly or a portion thereof defines a flexible, reusable bag that is sized and shaped to substantially fill the space provided by a flange assembly. Various connectors and retainers are additionally provided to repeatedly seal the container assemblies to both form a closed and an open configuration.

According to another aspect of the present disclosure, a method of operating a system for pumping milk into a storage container includes one or more of: providing the system comprising a skin contact member configured to form a seal with the breast, a conduit in fluid communication with and connected to the skin contact member; a driving mechanism including a compression member configured to compress and allow decompression of the conduit in response to inward and outward movements of the compression member, a sensor, and a controller configured to control operation of the driving mechanism; sealing the skin contact member to the breast; operating the driving mechanism to generate predetermined pressure cycles within the conduit; monitoring by the controller of at least one of position and speed of movement of the compression member relative to the conduit; measuring or calculating pressure within the conduit; maintaining or modifying motion of the compression member as needed, based upon feedback from the calculated pressure and at least one of position and speed of movement of the compression member, to ensure that the predetermined pressure cycles continue to be generated.

These and other features of the disclosure will become apparent to those persons skilled in the art upon reading the details of the systems and methods as more fully described below.

### BRIEF DESCRIPTION OF THE DRAWINGS

FIG. 1A shows a perspective view of a breast pump system according to an embodiment of the present disclosure.

FIG. 1B shows a rear view of the system of FIG. 1.

FIGS. 2A-B are perspective views, depicting placing a container assembly within a breast pump system.

FIG. 3 is a front view, depicting one approach to a container assembly body.

FIG. 4 is a front view, depicting another approach to a container assembly body.

FIGS. 5A-C are front views, depicting areas for making notations on a body of a container assembly.

FIGS. 6A-G are front views, depicting various approaches to container assemblies with spouts.

FIG. 7 is a front view, depicting a container assembly with a vent.

FIG. 8A is a front view, depicting a container assembly with additional sealed portions.

FIG. 8B is a front view, depicting another container assembly with additional sealed portions.

FIG. 9 is a front view, depicting a container assembly with alternative additional sealed portions.

FIG. 10 is partial view, depicting a portion of a container assembly with additional sealed portions.

FIG. 11 is partial view, depicting a portion of another container assembly with additional sealed portions.

FIG. 12A is a front view, depicting a further embodiment of a container assembly with dual film valve structure.

FIG. 12B is a cross-sectional view, depicting a closed film valve structure.

FIG. 12C is a cross-sectional view, depicting an open film valve structure.

FIG. 13A is a front view, depicting a fitment including an inverted boss.

FIG. 13B is a side view, depicting the fitment of FIG. 13A.

FIG. 13C-E are perspective views, depicting the fitment of FIG. 13A attached to a container assembly.

FIG. 14A is a perspective view, depicting another embodiment of a fitment.

FIG. 14B is a partial cross-sectional view, depicting the fitment of FIG. 14A.

FIG. 14C is a cross-sectional view, depicting the fitment of FIG. 14A inserted in a container assembly.

FIG. 14D is a cross-sectional view, depicting the fitment of FIG. 14A inserted in a container assembly in another configuration.

FIG. 15A is a perspective view, depicting another fitment embodiment.

FIG. 15B is a front view, depicting yet another fitment embodiment.

FIG. 15C is a perspective view, depicting still yet another fitment embodiment.

FIG. 15D is a front view, depicting a further approach to a fitment.

FIGS. 15E-K are various views, depicting yet further approaches to fitment assemblies.

FIGS. 15L-P are various further views, depicting details of yet other approaches to fitment assemblies.

FIGS. 16A-B are enlarged views, depicting a portion of container assemblies.

FIG. 17A is a front view, depicting a plug structure attached to a container assembly.

FIG. 17 B is a front view, depicting the plug structure of FIG. 17B placed into a fitment.

FIG. 18 is a front view, depicting another approach to sealing structure for a container assembly.

FIG. 19A is a front view, depicting one approach to a clip structure.

FIG. 19B is a perspective view, depicting the clip structure of FIG. 19A.

FIG. 19C is a front view, depicting another approach to a clip structure.

FIG. 20A is a perspective view, depicting fitment structure.

FIG. 20B is a perspective view, depicting a plug.

FIG. 20C is a cross-sectional view, depicting the plug of FIG. 20B inserted into the fitment structure of FIG. 20A.

FIG. 21A is a cross-sectional view, depicting a first embodiment of a mating connector.

FIG. 21B is a cross-sectional view, depicting a second embodiment of a mating connector.

FIG. 22A is a front view, depicting a container assembly with another fitment and plug arrangement.

FIG. 22B is a front view, depicting yet another fitment and plug arrangement for a container assembly.

FIG. 23A is a front view, depicting one embodiment of a flat fitment.

FIG. 23B is a front view, depicting another embodiment of a flat fitment.

FIG. 24A is an exploded view, depicting one embodiment of a reusable container assembly.

FIG. 24B is an exploded view, depicting another embodiment of a reusable container assembly.

FIG. 24C is an exploded view, depicting yet another embodiment of a reusable container assembly.

FIGS. 25A-B are exploded views, depicting a reusable container assembly that is split laterally.

FIG. 26 is an exploded view, depicting another approach to a laterally sectioned container assembly.

FIGS. 27A-B are perspective and exploded views, depicting yet another approach to a container assembly.

FIG. 28 is an exploded view, depicting a reusable container assembly with a keyed assembly feature.

FIG. 29 is an exploded view, depicting another approach to a reusable container assembly including a plurality of subcomponents.

FIGS. 30A-B are cross-sectional and perspective views, depicting a container assembly with an expansible portion.

FIG. 31 is a perspective view, depicting a reusable container assembly formed integral with a flange.

FIGS. 32A-B are exploded views, depicting additional approaches to container assemblies formed integral with a flange.

FIGS. 33A-F are perspective and exploded views, depicting yet further approaches to container assemblies formed integral with a flange.

FIG. 34 is a perspective views, depicting a flange with a rotating locking feature.

FIGS. 35A-B are perspective view, depicting a rotating attachment structure engaging a flange.

FIGS. 36A-J are various views, depicting approaches to flexible container assemblies.

FIGS. 37A-C are perspective views, depicting a fitment attaching to a container assembly.

FIGS. 38A-K are various views, depicting alternative approaches to spouts for a container assembly.

FIGS. 39A-H are various views, depicting alternative approaches to caps and spouts for a container assembly.

FIGS. 40A-K are various views, depicting alternative approaches to vents and venting of container assemblies.

### DETAILED DESCRIPTION OF THE DISCLOSURE

Before the present systems and methods are described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a sensor” includes a plurality of such sensors and reference to “the pump” includes reference to one or more pumps and equivalents thereof known to those skilled in the art, and so forth.

The publications discussed herein are provided solely for their disclosure prior to the filing date of the present application. The dates of publication provided may be different from the actual publication dates which may need to be independently confirmed.

Various details of the present system can be found in PCT Application Nos. PCT/US15/41257, PCT/US15/41271, PCT/US15/41277, and PCT/US15/41285 each filed Jul. 21, 2015, PCT/US15/50340 filed Sep. 16/2015, PCT/US/17/17112 filed Feb. 9, 2017, and PCT/US17/17212 filed Feb. 9, 2017, each of which are hereby incorporated herein, in their entireties, by reference thereto.

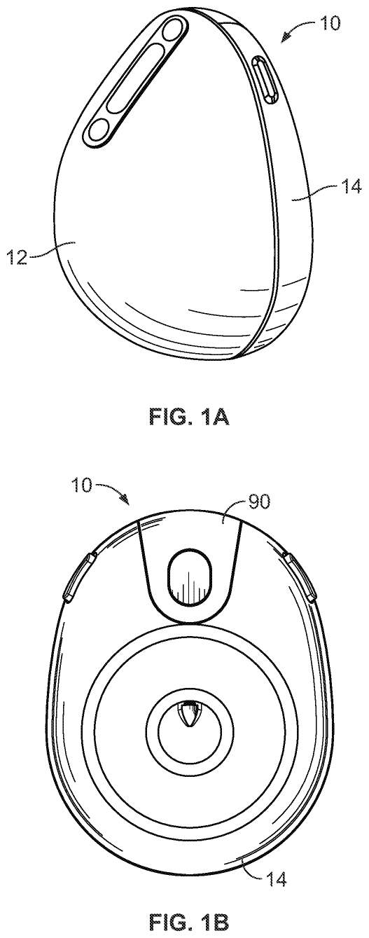

FIGS. 1A-B are perspective and back views of a breast pump system 10 according to an embodiment of the present disclosure. The breast pump system 10 can include one or more of the below introduced or described features or functions, or a combination thereof. The housing or outer shell 12 of system 10 can be shaped and configured to be contoured to the breast of a user and to thus provide a more natural appearance when under the clothing of the user. As can be appreciated from the figures, the system can define a natural breast profile. The natural breast profile is contemplated to fit comfortably and conveniently into a bra of a user and to present a natural look. Extending from the base are curved surfaces having asymmetric patterns. Moreover, like natural breasts, the profile of the device or system is contemplated to define one or more asymmetric curves and off-center inertial centers. Various natural breast shapes can be provided to choose from to the tastes and needs of a user. An opposite side of the pump system 10 is configured with a flange 14 which is sized and shaped to engage a breast of a user. The flange 14 is contoured to comfortably fit against a wide range of user's bodies and to provide structure for sealingly engaging with breast tissue.

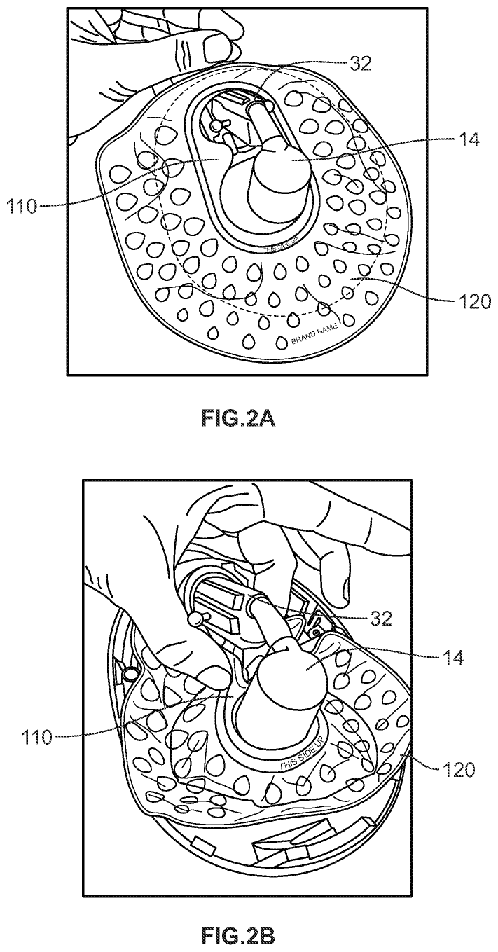

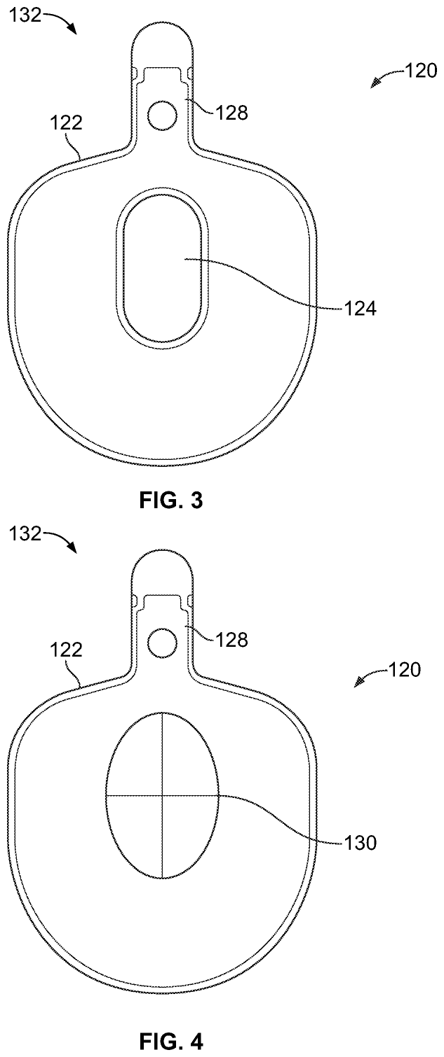

Various steps in loading a container assembly 120 into the system 10 is shown in FIGS. 2A-B. In a first step (FIG. 2A), the flange 14 is removed from engagement with the remainder of the system 10. Attached to the flange is the flex conduit or tube 32. A central portion of the container assembly 120 is placed over a central projection 110 of the flange 14 and the flex conduit or tube 32. Next, the user can pinch the container assembly and configure it under the flex conduit or tube 32 followed by tucking the container 120 into the flange 14. The opening 132 to an interior of the container assembly 120 (See FIGS. 2 and 3 for example) is fluidly connected to the flex conduit or tube 32 such as via the pump outlet 138. Once connected the door assembly 90 can be rotated over the flex conduit or tube assembly 82 and pump outlet connection (See FIG. 1A) to provide support and a robust engagement between the parts.

It is contemplated that the door assembly 90 (See FIGS. 1B) can be employed to both provide a continuous contour of the flange 14 for engaging a user's breast as well as to support the engagement of the container assembly 120 with the system 10. Thus, the door assembly 90 can be configured to pivot with respect to the flange 14, and employed to close the system 10 as it is snapped over and closes the pump. With this approach, the container assembly 120 is securely sandwiched within the door assembly 90.

As described in connection with the embodiments presented below, the system 10 can be configured to pump into a container assembly. In one or more approaches, the container assembly can include various embodiments of a fitment that can be attached to or integrally formed with a bag assembly such that it is associated with or molded along with one or more sheets defining bag structure or other structure defining an entrance to an interior of a container assembly. Such fitments are configured to mate with pumping structure and in particular with a flex conduit or other conduit through which milk is pumped. Sealing and/or airtight structures can hold liquids and gases without permitting a leak.

In at least one embodiment, the pressure at which a valve (not shown) configured between the pump system and the container assembly opens to allow flow into the milk collection container is about 25 mm Hg. The valve can be configured and designed such that it allows fluid to flow through it when the pressure is positive, e.g., about 25 mm Hg, or some other predesigned “opening pressure”. A valve can also be included in the container assembly itself.

Turning now to FIG. 3, one embodiment of a sealed or sealable container assembly 120 is shown. In one particular embodiment, the container assembly 120 can be formed from about two 2.5-3.0 mil sheets of material that is joined (i.e., welded) together along a perimeter 122 of the assembly, and can be sized to retain up to 3.5 ounces, or alternatively 8 ounces or more of fluid. A body of the container assembly (e.g. 4 oz, 8 oz) is shaped to fit within flange 14 (such as 24 mm, 27 mm sizes) and includes a generally oval opening 124 created by an interior seal. A narrow neck portion 128 is centrally positioned and extends longitudinally away from the central opening 124. The container assembly 120 can be re-sealable, re-usable, include larger or smaller openings, or include spout structure for pouring contents. With rounded out shoulder and inner opening, the volume capacity can be approximately 6 ounces or more.

Moreover, in one particular embodiment, the container assembly 120 can be made from polyethylene and can be bisphenol A free, as well as food grade material. It is freezable without tearing and withstand approximately −80 degree Celsius temperatures. Additionally, tensile strength can be from 2300-2900 psi and tear strength from 440-600 psi, with a water vapor transmission rate max of about 0.5 g/100 in2/24 hrs and an oxygen transfer rate of about 150 cc/100 in2/24 hrs. In alternate embodiments, the material of the container assembly can include Gore-Tex or Tyvek, for example. Such alternative materials can permit out-gasing such that any air that is pumped into the container assembly will escape through the material while the container assembly retains the fluid. In this specific regard, other vents or approaches to venting the system can be incorporated into one or more embodiments. Thus, self-venting of the container assembly or active venting while using the pump system or after use can be accommodated.

As shown in FIG. 4, the container assembly 120 can lack the central oval opening and alternatively include a central portion defined by scored material or material that is slit such as defining a cross 130 configured to open when the container assembly is placed into a breast pump system. This approach lends itself to ease of manufacturing since the central opening need not be created and material therefrom need not be removed. In the embodiments presented in various figures, the opening 132 to an interior of the assembly is located in the neck portion.

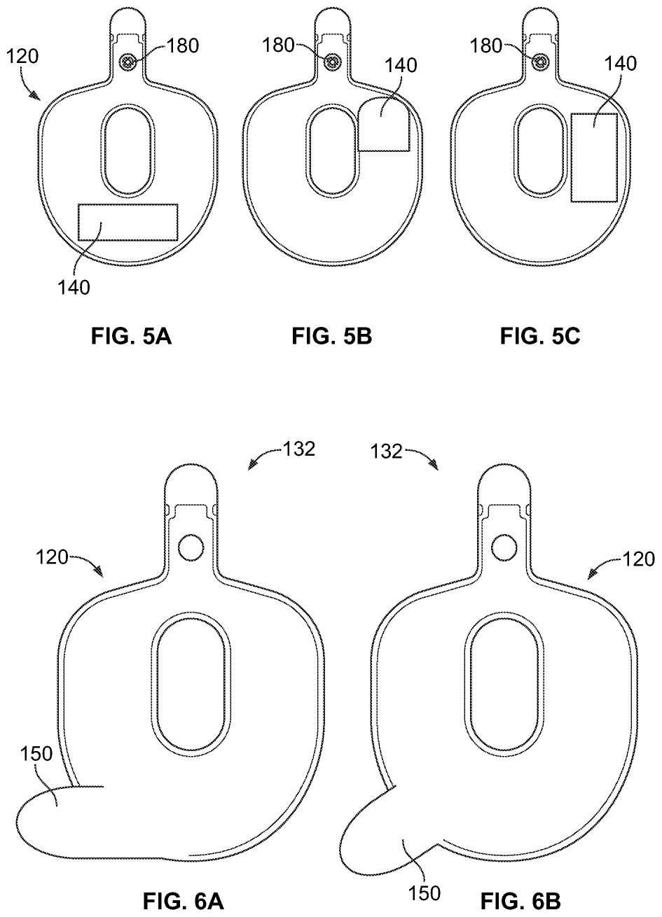

As shown in FIGS. 5A-C, the container assembly also includes areas 140 designated to define a writeable space. Various information such as that related to pumping sessions can be written in such areas, and such areas can be provided anywhere on the container assembly. Notably, this and other printing on the container assembly can be seen by the user through a transparent or otherwise conveniently configured flange, the same being useful where alignment indicators are provided on the container assembly. For example, a colored ring around the opening to the container assembly can highlight an area around the flange tunnel and where the breast needs to enter the flange.

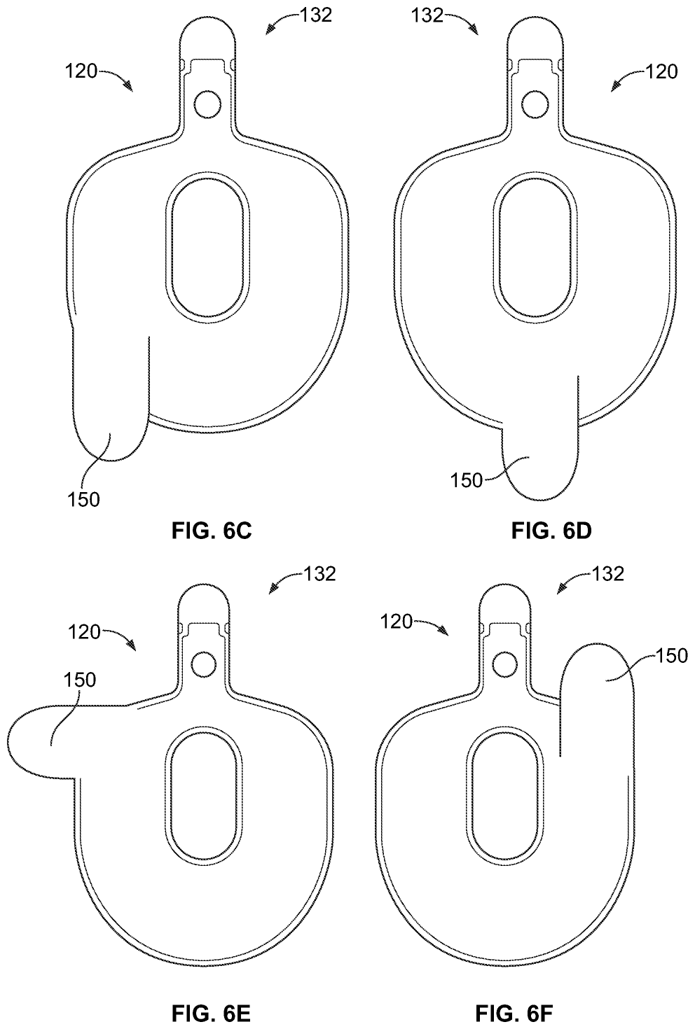

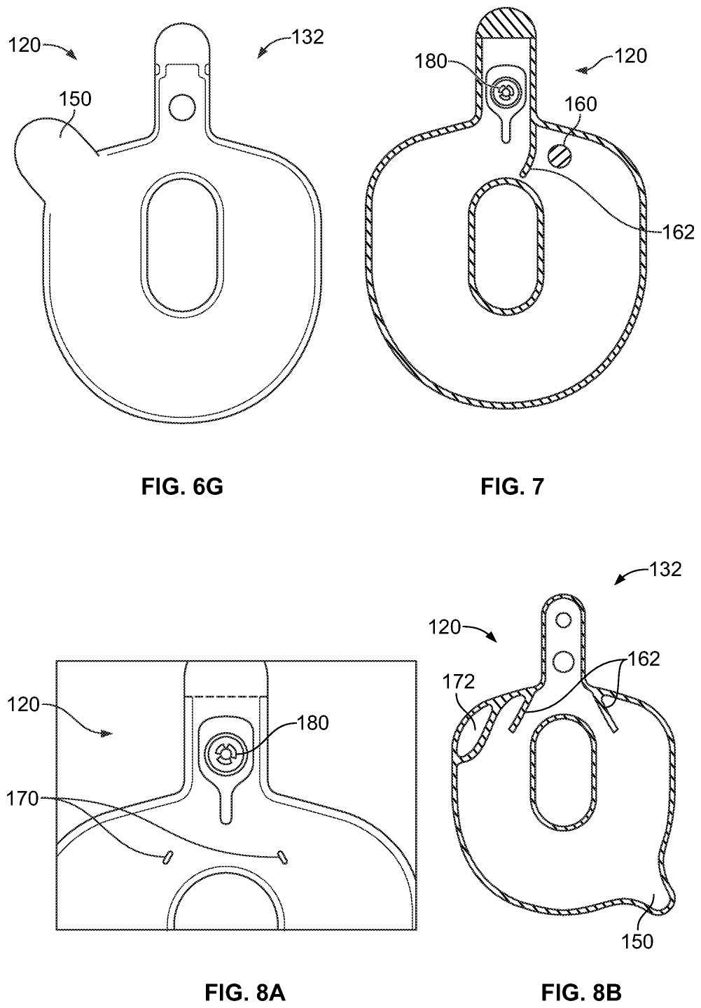

Referring to FIGS. 6A-G, in various embodiments, the container assembly 120 further includes an additional spout 150 for pouring collected milk. Such spouts 150 can be placed at various positions about the body of the container assembly. Exits for the spouts can be created by cutting spout material, or the spout can additionally include scoring or a resealable opening.

Additionally or alternatively, the container assembly 120 further includes one or more vents 160, such as a Porex vent or an equivalent thereto (See FIG. 7). The vent 160 releases air while milk is pumped into the container assembly 120 where there is a one-way valve placed either attached or integral to the breast pump system or the container. In one embodiment, once the vent 160 is exposed to moisture or is otherwise clogged, it no longer allows the passage of air or fluid. However, should the vent become again dry, it can still allow some air flow. Thus, should the vent 160 be exposed to moisture, the vent 160 seals to avoid a fluid leak. This particular container assembly 120 is further characterized by an internal seal 162 that facilitates operation and functioning of the vent 160 by diverting collected milk and air in a desired fashion within the container assembly such as for preserving the function of a porex patch for example, which needs to stay dry for venting air. Accordingly, in one embodiment, the internal seal 162 diverts fluid from the vent so the vent remains functional and continues to allow the passage of air out of the container assembly. Consequently, the user can pump more fluid into the container assembly 120 without the container volume being compromised of air and the container assembly 120 defines a lower profile when stored. Moreover, this approach results in reducing the number of container assemblies used in a single pumping session, especially for new breast pump system users who may pump more air into the system due to multiple re-alignments with the breast.

In another approach (FIG. 8A), the container assembly 120 includes additional seal points or tack welds in shoulders of the container assembly 120. Such tack welds 170 increase adhesion between container film layers to prevent fluid backflow and spilling of milk during removal and handling of the container 120 after completing a pumping session. Moreover, such tack welds or other adhesion between layers does not adversely impede fluid flow in the container due to positive pressure of the pump system, but does restrict an outward flow and hence spilling.

Turning to FIG. 8B, there is shown a container assembly 120 that includes a pair of internal seals 162 configured to divert collected milk away from the entry to the container assembly. This embodiment further includes a sealed portion 172 that is arranged to not accept milk but is otherwise provided as an area for writing thereupon by the user. This embodiment further includes a spout 150 configured into a lower portion of the container assembly 120.

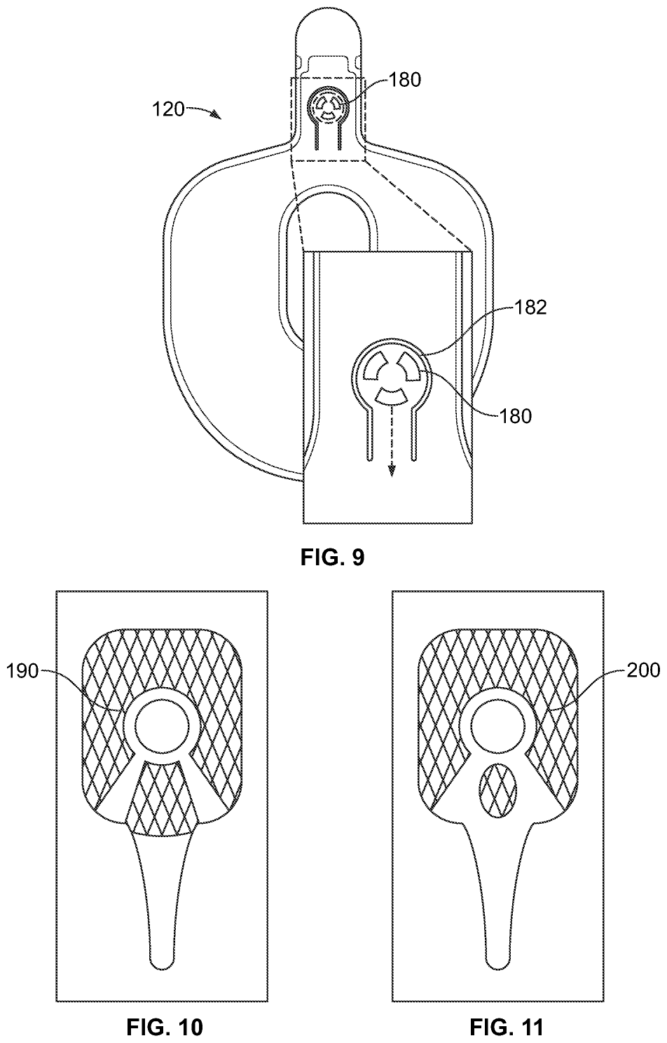

As shown in FIGS. 9-11, additional or alternative portions of the container assembly can be sealed or tack welded together. In one approach (FIG. 9), container film is sealed against a rear portion of a fitment 180. Here, a generally key-hole shaped path 182 about the fitment is created to define a fluid path from a flex conduit through the fitment 180 and into the body of the container assembly 120. Alternate sealing patterns 190, 200 (FIGS. 10-11) are also alternatively or additionally employed to create desired paths out of the fitment 180 and into the body of the container assembly 120. Such sealing patters are adjusted or configured to achieve desired film tension and breast pump purge efficiency.

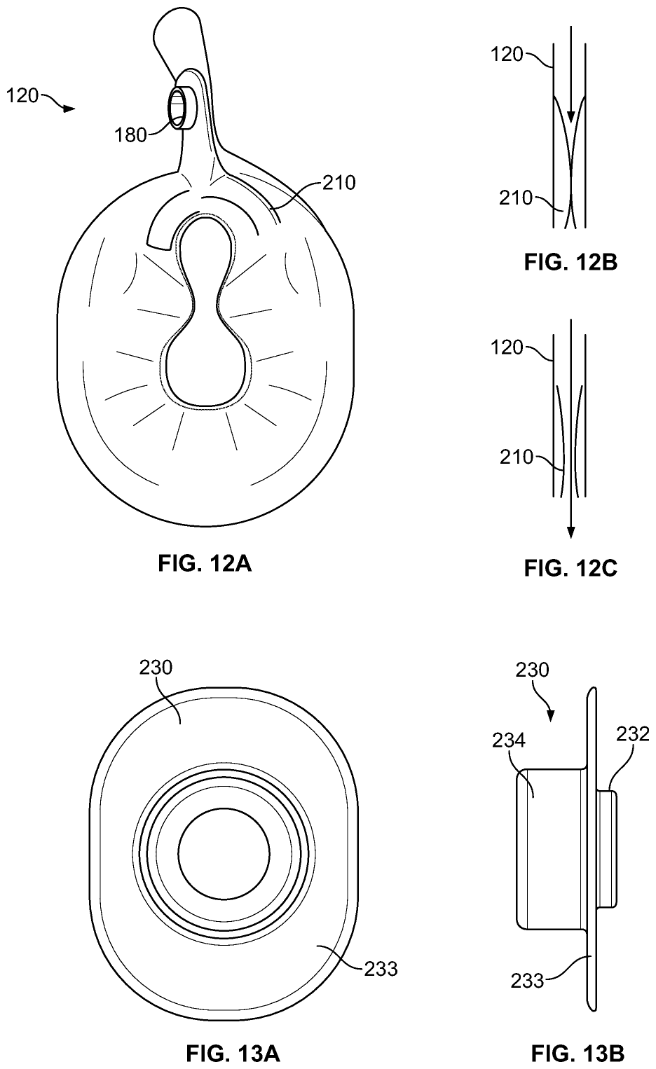

In another embodiment, as shown in FIGS. 12A-C, the container assembly 120 includes a dual film valve 210. The dual film valve 210 is formed from additional film layers sealed in an interior of the container. Various patterns of sealed layers can be employed. In one specific embodiment, the layers define a wishbone-like geometry to accommodate container assembly geometry. The film valve 210 is sealed to a back side of the fitment 180, and fluid flows through the wishbone structure and exits into the body of the container assembly 120. As fluid is purged through the fitment and exits the dual film valve or where fluid is being pushed into the container assembly, the layers separate and allow the passage of fluid within the container assembly (FIG. 12C). In its resting state or under passive pressure (FIG. 12B), the film layers come together and act as a valve (film layers remain sealed under tension). The geometry of the dual layer can be modified to adjust the performance of the dual layer valve, such as for desired flow restriction or efficiency as a valve.

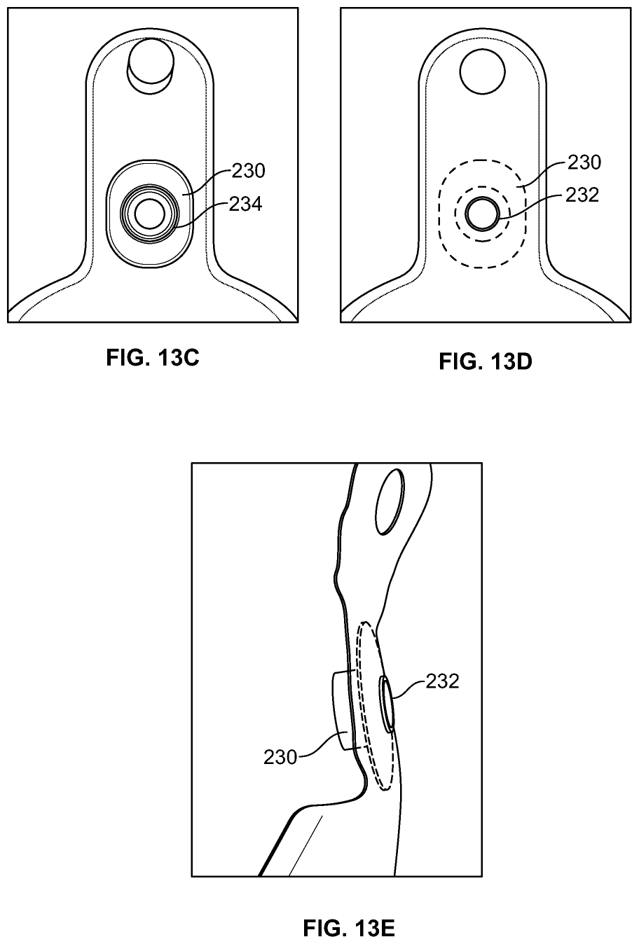

Turning now to FIGS. 13A-E, there is shown another approach to a fitment 230 for a container assembly 120. This fitment 230 includes an inverted fitment boss 232 that projects from a base 233 and is configured to increase or provide sufficient film tension between the boss 232 and the interior bag surface, and discourages fluid from flowing back out from the container (FIG. 13E). Projecting from the opposite side of the base 233 is a cylindrical boss 234 that defines the passage into the container assembly 120. As so configured, the inverted boss 232 functions to reduce fluid backflow and the spilling of collected milk. Accordingly, the user is provided with more flexibility with removal and handling of the container assembly 120 after completing a pumping session while minimizing the potential for spilled milk.

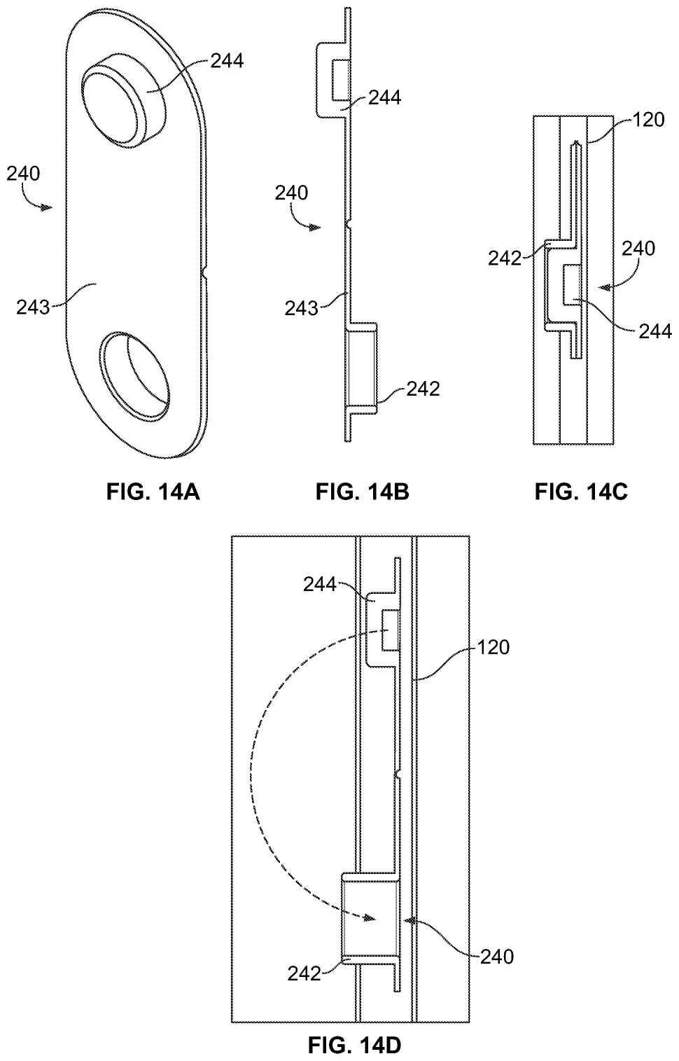

Turning now to FIGS. 14A-D, there is shown a dual boss fitment 240. A first boss 242 extends perpendicularly from a generally oval base 243 and includes a thru-hole that defines the passage into the container assembly 120. A second boss 244 is spaced from the first boss and projects from an opposite side of the base 243. In this embodiment, the base 243 can be folded via a hinge or equivalent structure so that the second boss 244 aligns with the first boss 242. As shown in FIG. 14C, the base is first folded so that bosses are so aligned when the dual boss fitment 240 is configured within the container assembly 120. Alternatively, as shown in FIG. 14D, the dual boss fitment is configured in a container assembly in an unfolded configuration, and then folded over so that the second boss 244 is engaged within the first boss 242, using the container film to create an interference fit. Thus, pinching the two bosses together functions to close the opening to the container assembly 120, where the second boss 244 operates as a cap. In such a closed configuration, the container assembly 120 is sealed and sterility is maintained until the container is assembled as part of a milk path.

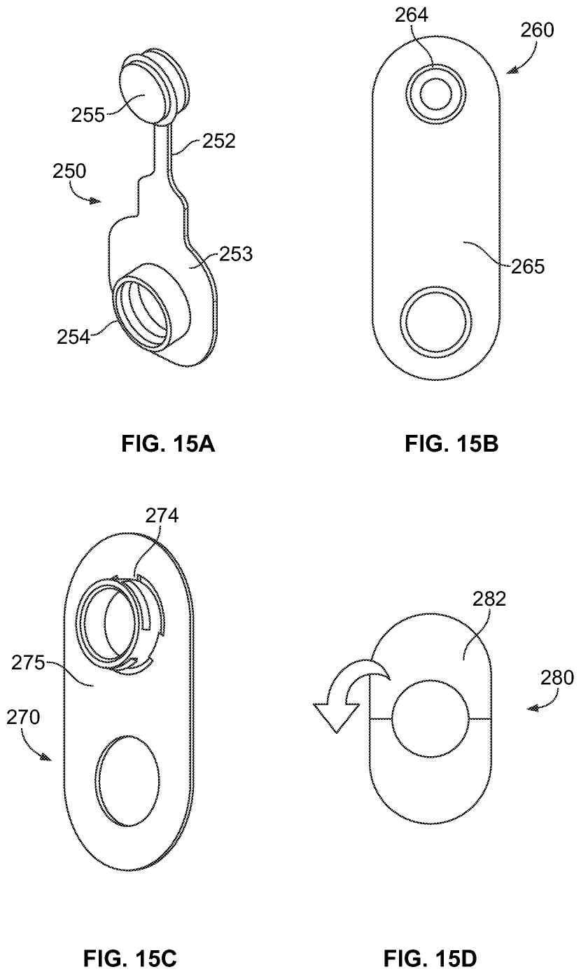

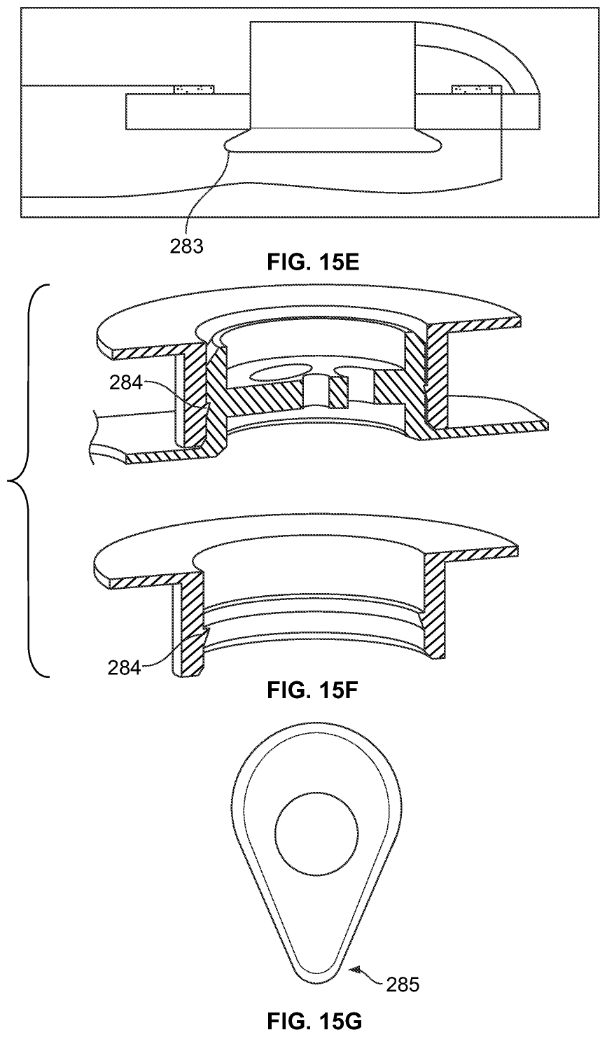

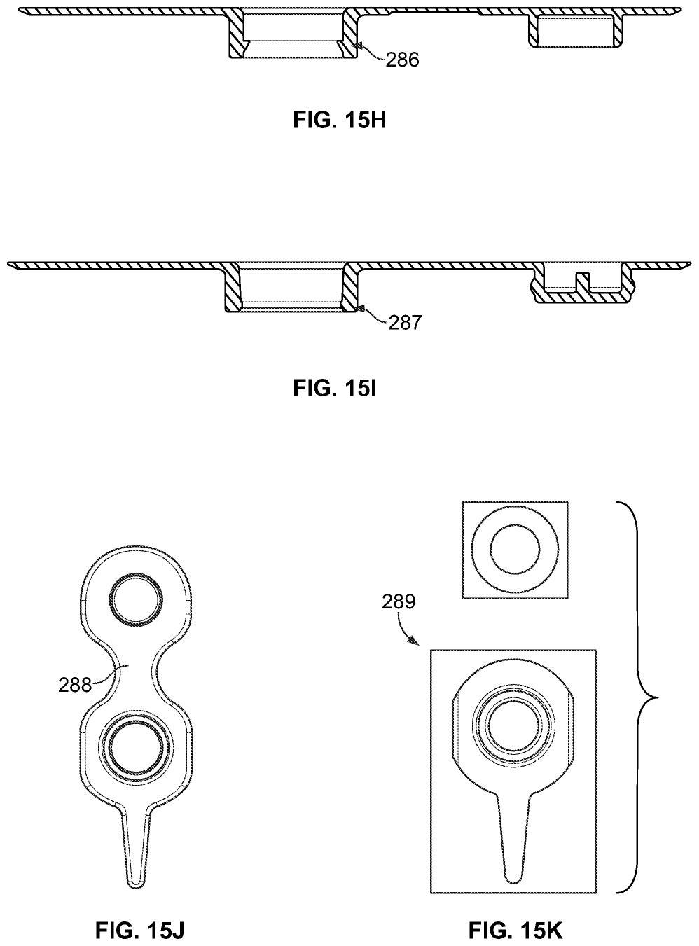

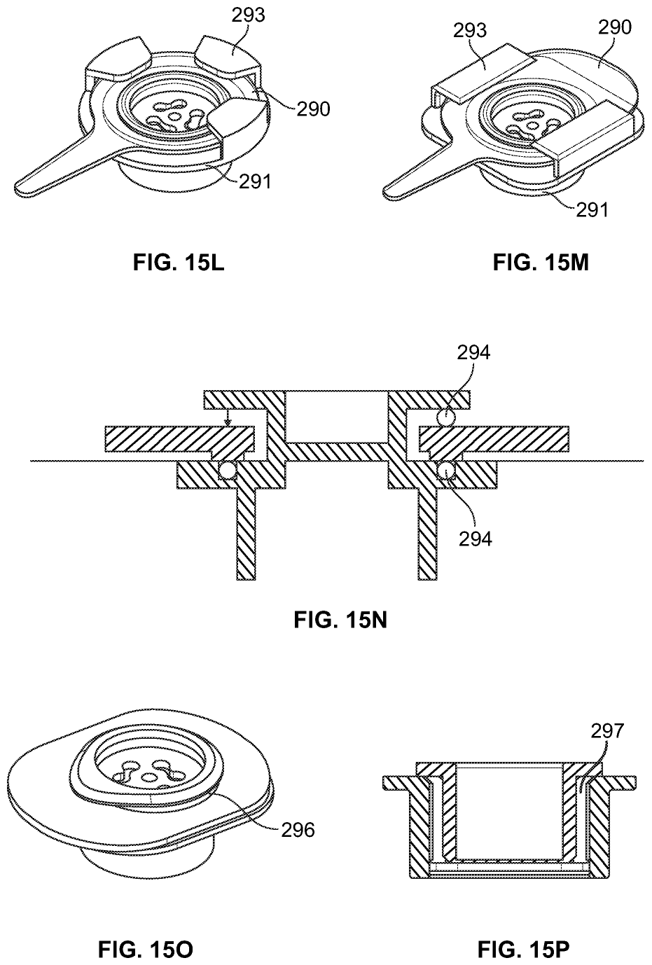

Alternative approaches to foldable fitments are shown in FIGS. 15A-D. The fitment 250 depicted in FIG. 15A is characterized by a leash structure 252 between a base 253 of a first boss 254 and a second plug boss 255. The fitments 260, 270 shown in FIGS. 15B and C include second bosses 264, 274 including through holes and generally oval bases 265, 275. In these approaches, a cap seal is created against the fitment via pressing or interference. The fitment approach 280 depicted in FIG. 15D includes a base 282 that folds along a line that divides a through hole formed in the base 282.

Further additional or alternative features of fitments are shown in FIGS. 15E-K. As depicted in FIG. 15E, a cap or plug can alternatively or additionally include an annular rib 283 that is configured to pop through a second, generally flat fitment to provide a sealing structure. Here, a cap would be configured on an outside of a bag film. FIG. 15F depicts a fitment including a sharp sealing internal barb 284 feature that can be included as additional or alternative structure in a fitment. When the barb 284 is pressed onto mating structure (each of which can be formed from LDPE), it functions to seal against an outer surface of the fitment. Further, as shown in FIG. 15G, a fitment can lack a tail and define a generally tear shape 285, the same functioning to permit milk flow while taking up less space. A snap tactile feedback between parts can also be incorporated into various fitments (FIG. 15H). That is, an annular internal barb 286 is configured to seal against mating structure. In one aspect, there is provided a generally hour-glass shape that allows for and facilitates this functionality. A related approach is shown in FIG. 15I, where a fitment can alternatively or additionally include a cap with an annular rib 287 that when inserted into a fitment boss, it pops past an annular undercut in the fitment boss to create a seal as well as provide both tactile and audible snap feedback. Additionally, the fitment assembly can also include a narrowed waist 288 connecting cap and fitment portions, the waist facilitating the folding of parts (See FIG. 15J), or alternatively, the pieces can be separated and oriented as the arrangement of the fitment and plug assembly 289 shown in FIG. 15K.

Turning now to FIGS. 15L-P, there are shown additional assemblies that leverage material interfaces as sealing structure to provide low profile fitments, self-contained compression between parts and assembly assistance. In one approach (FIG. 15L-M), a short flat fitment boss 290 is configured to engage and seal against a one way valve assembly 291 that includes a second shot section formed from TPE material. As such, the container assembly can assume a low profile structure and be configured to seal with a valve assembly that is separate and distinct from the container assembly. This low profile approach allows for the container assembly to occupy less space both during shipping and storage. Tabs 293 are provided on the one way valve assembly 291 and can be arranged in circular (FIG. 15L) or rectangular (FIG. 15M) patterns. Alternatively or additionally, a flat fitment 290 is sealed against one or more o-rings 294 (FIG. 15N). Here, the valve assembly structure provides compression onto the flat fitment to push it against the o-rings 294 and form a face seal. Moreover, alternatively or additionally, an inner diameter 295 of a flat fitment is arranged to seal against a second shot TPE surface forming an outer diameter of a valve assembly boss 296 (FIG. 15O). Accordingly, compression for a seal is provided by radial interference. In another aspect, the valve assembly boss can have a TPE outer diameter and can be angled or have a flat top, and the flat fitment can further include an inverted boss to aid flow of milk into the container assembly.

In further aspects, one or more components of a fitment can be formed from layers of TPE/LDPE/TPE or similar variations so that the TPE parts are utilized as sealing material with mating parts. Thus, in various aspects, this allows for an all-polypropylene valve assembly. Moreover, the fitment boss of a valve assembly can be formed from TPE that sealingly engages with an LDPE fitment so that there is provided a hard to soft material radial seal. The fitment alternatively or additionally is stamped/die-cut from TPE material where geometric sub-structure provides flexing where necessary and more rigid sections for support stiffness, as stiffness is necessary for a seal, but flex is required to decrease user assembly forces. The valve assembly can alternatively or additionally include over-molded TPE surfaces that interface and seal against a container fitment. Furthermore, a flat fitment can include a face that seals with a TPE face of a valve assembly, the combination creating a seal as a system door is closed over the parts. A TPE cylindrical part 297 also can be placed between mating parts (See FIG. 15N), where there is initially an annular space about the TPE part 297 until the fitment is placed within the TPE part to thereby create the necessary seal and close the space. Also, in another aspect, there is provided a Santoprene valve assembly part that naturally rests so that there is a gap thereabout, and upon insertion of a second part, the Santoprene part is pushed outwardly to close the gap and create a seal.

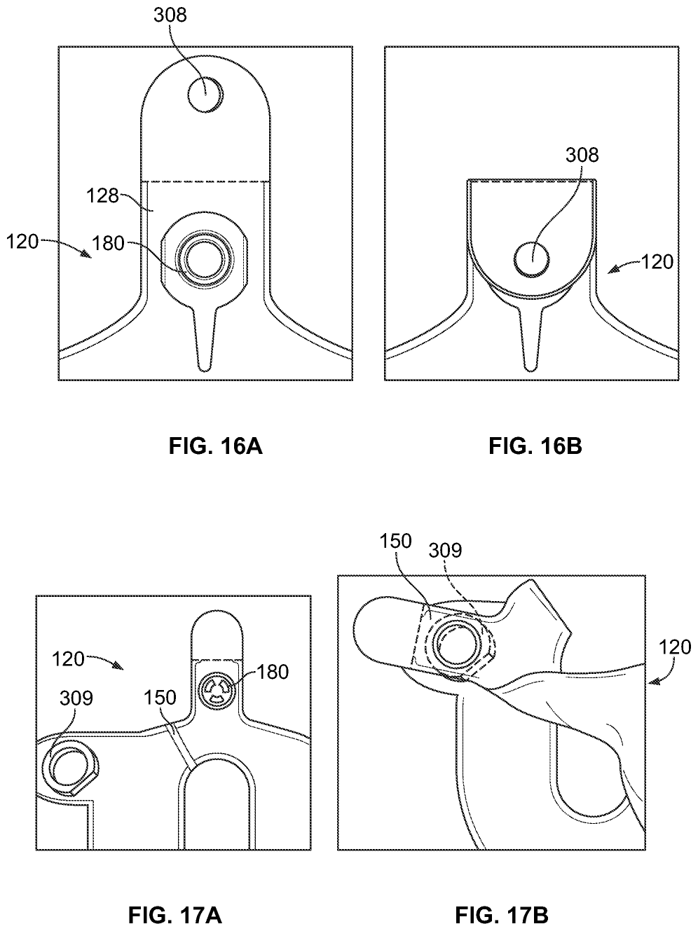

In one specific approach (FIGS. 16A-B), there is provided a container assembly 120 configured with a rubber plug 308 sized and shaped to be received within a fitment 180 attached or formed in the neck 128 of the container assembly 120. The neck assembly 128 is folded over so that the plug 308 is sealingly received in the fitment 180. In a related approach as shown in FIGS. 17A-B, the container assembly 120 embodies a spout structure 150 equipped with a rubber ring 309 that is configured to seal against an outer surface of the fitment 180 when the spout 150 is folded into position. It is to be understood that various locations of plugs and fitments are possible. For example, the plug can alternatively or additionally be positioned on a body of the container removed from a neck portion. In these and other approaches to fitments, internal and/or external structures of mating parts can assume unique and/or non-round engaging surfaces and structures so that proper alignment between parts is facilitated. Moreover, one or more of the fitment and plug can include an annular snap that engages an undercut shelf on its mating part.

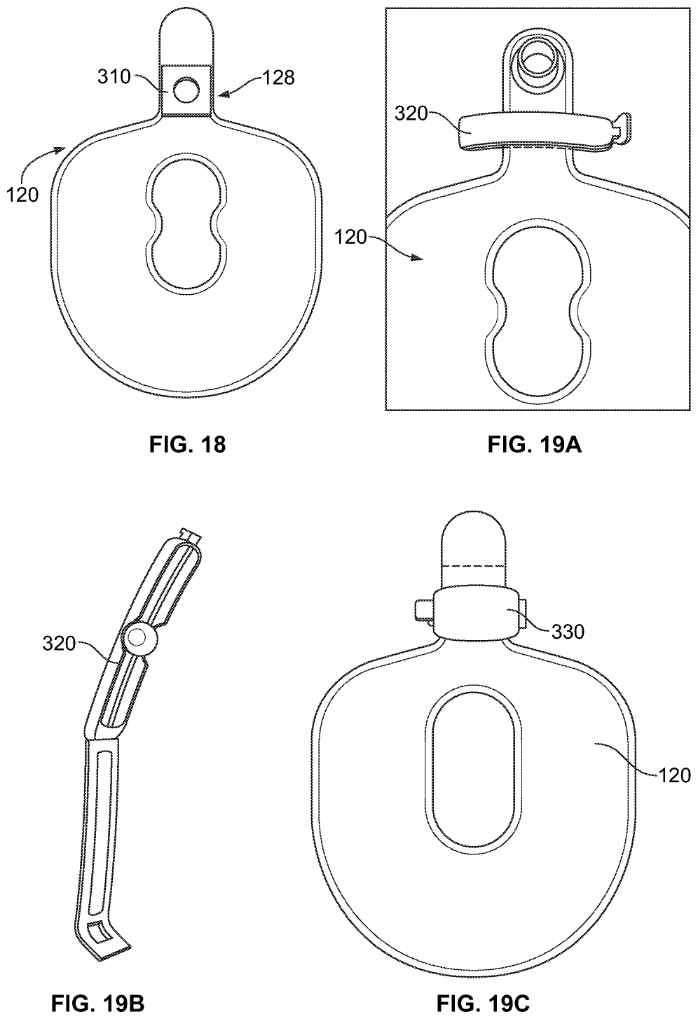

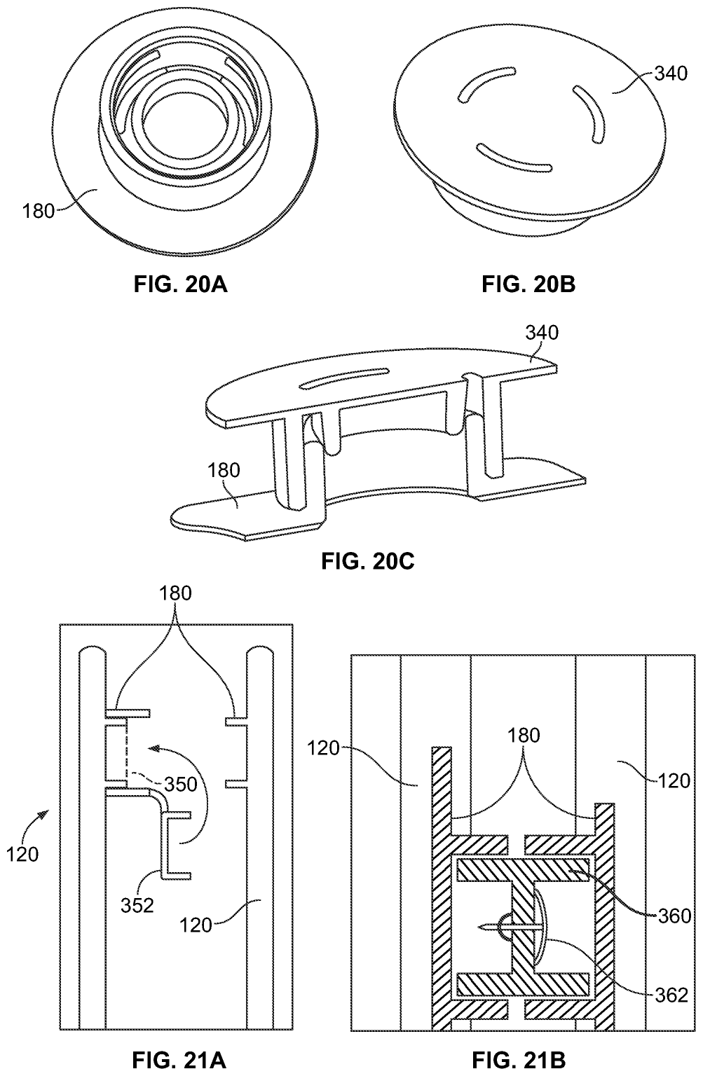

As shown in FIGS. 18A-B, the neck 128 of the container assembly 120 can itself also be used to seal the container assembly 120 where the fitment is additionally equipped with an adhesive foam pad 310 that includes a removable adhesive liner. Here, the neck 128 is folded over the foam pad 310 to create the seal. Additionally, in yet other approaches (FIGS. 19A-C), various foldable clips 320, 330 can when closed, be provided to engage and seal the container assembly 120. Moreover (FIG. 20), a stand-alone cap 340 can additionally or alternatively be provided to mate with a fitment 120 to function as a seal.

Turning now to FIGS. 21A-B, there are shown mating connectors 350, 360 that are configured to provide mating structure between two container assemblies 120 so that milk can be transferred from one container to another. As shown in FIG. 21A, the mating connector 350 is sized and shaped to sealingly fit about exterior surfaces of fitments 180 and further includes a cap 352 that is configured to close the mating connector. The mating connector 360 shown in FIG. 21B can include a one-way valve 362 that functions as a seal, and can be sized and shaped to fit within an interior of the fitments. Alternatively, this connector can lack the one-way valve and thus define structure for transferring liquid between container assemblies. These structures can be separate pieces or can be integrated into an existing system component such as the fitment. By using such an arrangement, a breast pump system user can then combine milk from multiple containers which helps to reduce the number of container requiring storage and allows the user to store specific volumes of milk in containers to assist in later feedings.

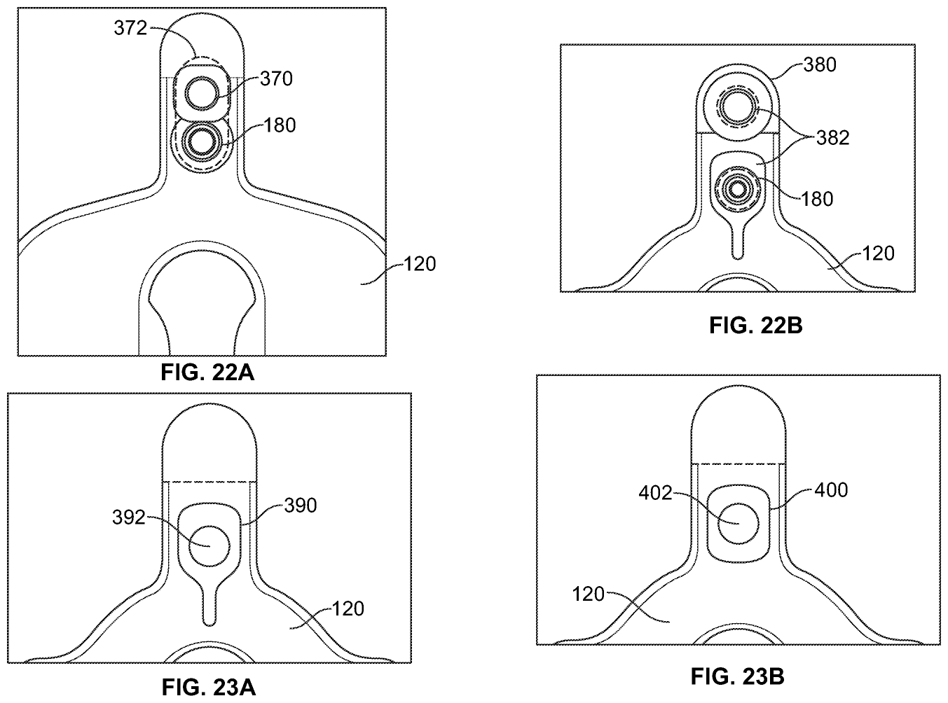

With reference to FIGS. 22A-B, there are shown additional embodiments of container assemblies 120 having fitments 180 and including plugs 370, 380 that are configured to be received in the fitments 180. In a first approach (FIG. 22A), the fitment 180 and plug 370 are arranged and attached to the container 120 within an oval opening 372 formed in the container assembly 120. The second approach (FIG. 22B), two separate holes 382 are formed in the container assembly 120 and size to individually receive the fitment 180 and plug 380.

FIGS. 23A-B illustrate container assemblies 120 that include flat fitments 390, 400. Such flat fitments 390, 400 are characterized by having a simple low profile construction that is configured like previous embodiments so that structure thereof is placed between container layers. Thru-holes 392, 402 formed in the fitments provide access to the interior of the container assembly 120.

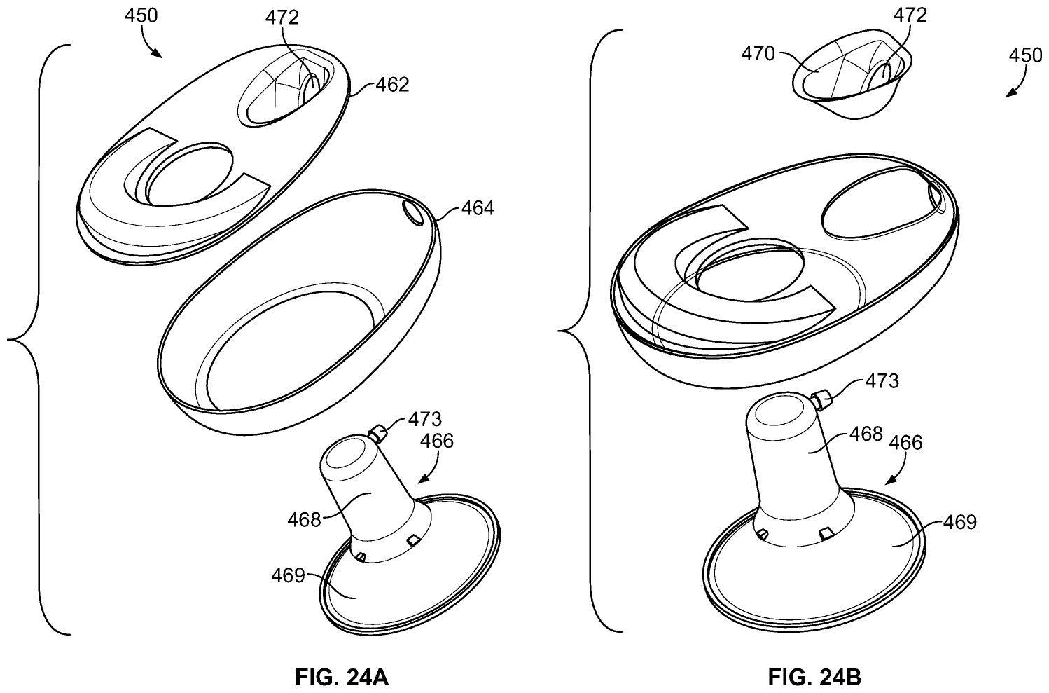

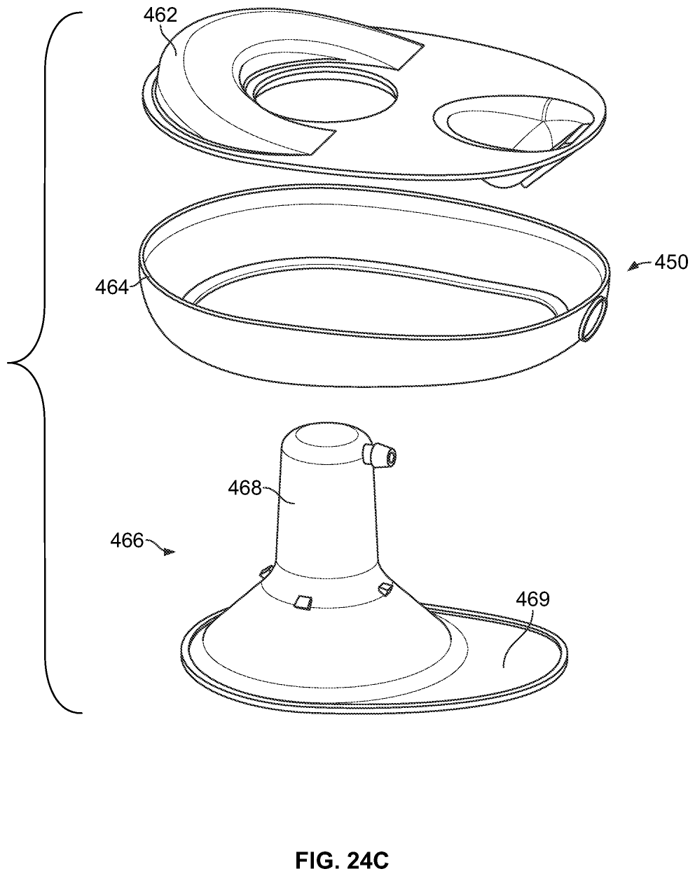

As stated, the container assemblies disclosed herein can be disposable or reusable and can be partially or entirely formed from one or more of rigid or flexible materials. Moreover, container assemblies can include breast interfacing structure and thus replace or incorporate a flange, or can be attached to a flange of a breast pump system. Thus, in various embodiments, the container assembly is configured to assemble with the breast pump and the pump is configured to recognize its presence prior to pumping such as via a hall effect sensor or valve structure. In certain approaches, the container assembly is compatible with multiple flanges and sizes. Each of the following disclosed features can define additional or alternative structures for each of the disclosed container assemblies. Turning now to FIGS. 24A-B, there is shown a reusable container assembly 450 formed from multiple parts. In a first approach, the container assembly 450 is defined by a first part 462 configured to be retained within an interior of an assembled breast pump system (See FIG. 1A), and a second part 464 each of which mating with a funnel 466, the second part 464 and funnel defining an outer surface of a breast pump system. The funnel 466 includes a nipple receiving portion 468 and replaces the flange presented in connection with previous disclosed embodiments. An outer surface of the nipple receiving portion 468 of the funnel 466 is configured to be removable from the first and second parts 462, 464. The outer surface of the nipple receiving portion 468 is sized and shaped to seal with an opening formed in the first part 462 and a generally circular base 469 of the funnel 466 seals with an opening in the second part 464, the three components cooperating to thereby define an enclosure for receiving milk pumped by the breast pump system. The container assembly 450 is generally longitudinally split with the first part 462 being connected to a second part 464 via welding or a seal along a perimeter of the two parts. The outer perimeter of the two parts match that of the breast system outer or external shell. The assembly can alternatively or additionally include a removeable plug 470 facilitating access to an interior of the container assembly 450 for cleaning and/or for pouring collected milk therefrom. The removable plug 470 or alternatively its corresponding structure in an assembly lacking the plug, includes an opening 472 sized and shaped to mate with or otherwise receive a flex conduit or tube and associated fitment (See FIG. 2A). This opening 472 can also be equipped with a fitment (not shown) for mating with the flex conduit. A second end of the fitment is sized and shaped to sealingly engage a nipple or extension 473 projecting from the exterior of the nipple receiving portion. In its assembled configuration, the container assembly 450 can be stored for later use and when disassembled, the parts can be conveniently washed for future use. In a related approach (FIG. 24C), the funnel 466 has a generally oval shaped base 469 that mates with corresponding structure or an opening of the second part 464. The oval base assists the user with properly assembling the funnel 466 to the first and second parts 462, 464.

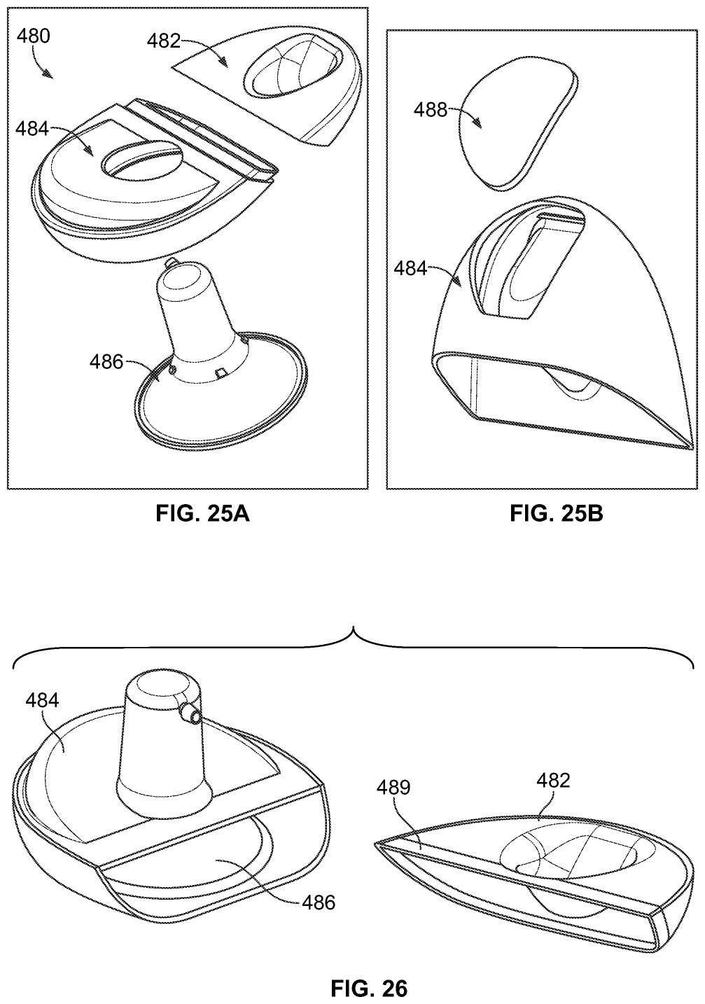

With reference to FIGS. 25A-B, there is shown another approach to a reusable container assembly 480. Here, the container assembly 480 is generally divided laterally and includes a first part 482 defining a top of the container assembly 480 and a second part 484 defining a bottom of the container assembly 480 that seals with or is welded to the first part 482. The assembly further includes a funnel 486 that mates with the first and second parts 482, 484 in a similar manner as described above to create a container for retaining milk and one that mates and completes a breast pump assembly. A plug 488 can be further provided to removably mate with the second part 484 so that access is provided to an interior of the container assembly. Further, as before, the container assembly is configured, sized and shaped to sealingly mate with a flex conduit or other structure through which milk is pumped. As shown in FIG. 26, structure for mating the first and second parts 482, 484 can include a sealing surface 489. In one approach, this structure can be more rigid than the rest of the first part 482 to provide additional strength for a robust fit or attachment. Other approaches to this and other connections to mating parts of a container assembly can include a Boston-shaker connection, a hinge and a latch arrangement or a detent and seal engagement. Moreover, here, the funnel and second part can define a single structure or multiple pieces.

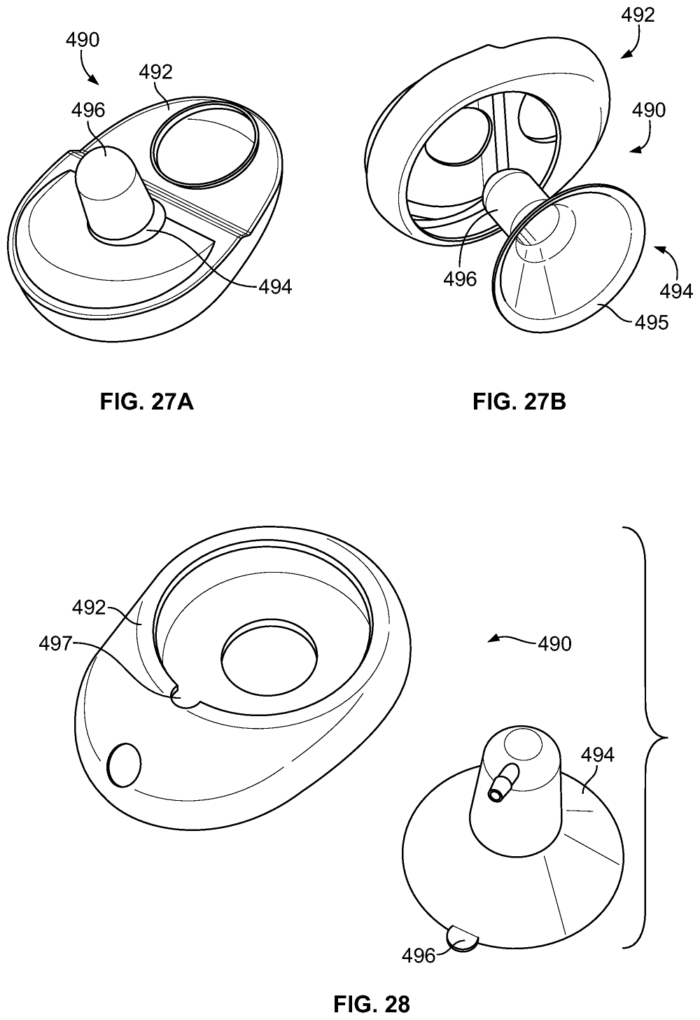

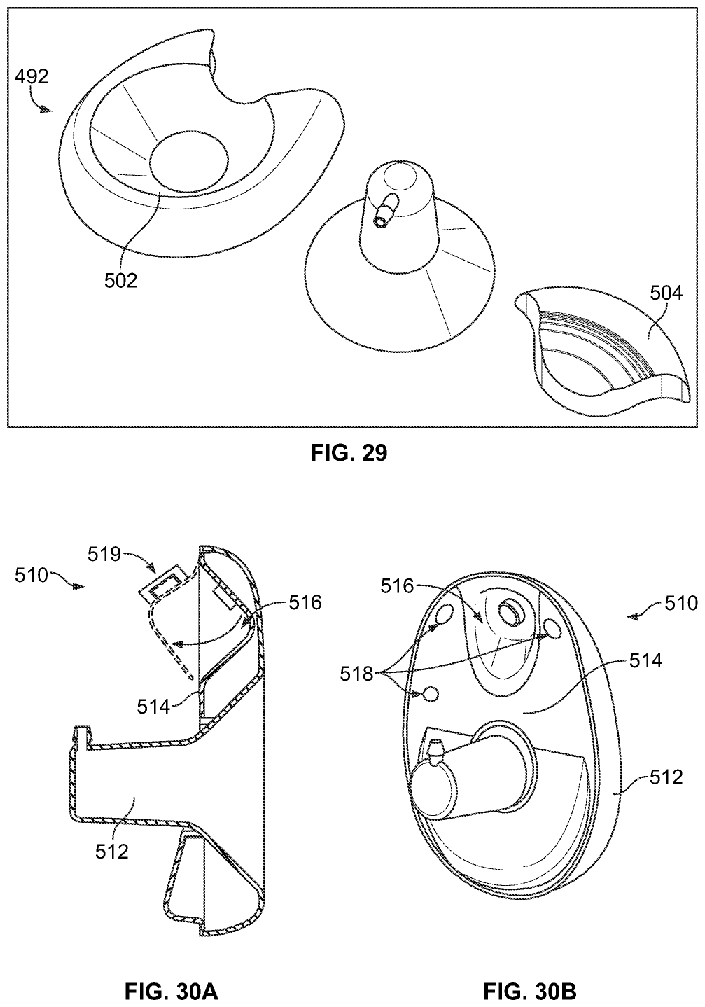

Turning to FIGS. 27A-B, the container assembly 490 can alternatively be formed from two pieces, a main body 492 and a funnel 494 that sealingly mates with the main body 492. The funnel includes a base 495 that mates with an opening formed in a first outer side of the main body 492 and an outer surface a nipple receiving portion of the funnel 494 mates with an opening formed in a second inner side of the main body 492 to form a container for receiving milk pumped by the breast pump system. When assembled, the container assembly 490 completes the breast pump system with an outer perimeter of the container assembly mating with the remainder of the breast pump assembly. Moreover, as before the container assembly 490 includes structure that mates with and receives a conduit that transports milk to the container assembly 490 and the funnel 494 and first outer side of the main body 492 define structure that engages the breast of the user. As shown in FIG. 28, the container assembly 490 can further embody a funnel 494 that includes tab 496 that mates with a recess 497 formed in the main body 492. This structural arrangement aids the user in properly assembling the funnel 494 to the main body 492. In yet another approach (FIG. 29), the main body 492 can be formed from two pieces, a bottom portion 502 and a top 504 that mate along a scalloped edge. Here, the top 504 can be removeable and sealed to the bottom portion 502.

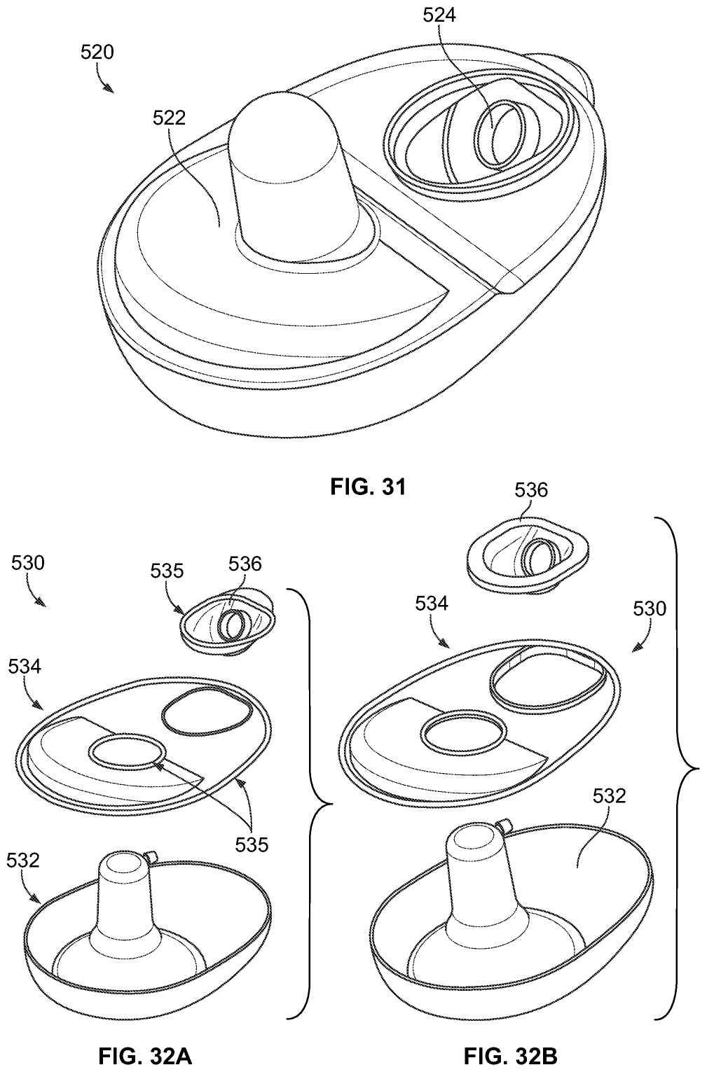

As shown in FIGS. 30A-B, the container assembly 510 can be defined by a flange 512 that is configured to mate with a cover or lid 514 that includes an elastomeric region for a bi-stable port area 516. There is sealing structure along an engagement perimeter between the flange 512 and the lid 514. The lid can additionally further include strike plates or hall effect actuator components 518 which facilitate communicating that the container assembly is connected to the remainder of the breast pump system. As best seen in FIG. 30A, the elastomeric region 516 can be placed into its expanded configuration to present a pouring structure which can be capped 519 for storing of milk received within the container assembly 510. The elastomeric region 516 further includes structure for mating with a conduit through which milk is pumped to the container assembly 510.

In a related approach (FIG. 31), a container assembly 520 is embodied in a two component assembly. A first part 522 incorporates flange and breast engaging structure and a bottom portion of the container assembly 520. A separate, removable cap and valve assembly 524 is configured to mate with the first part to complete the container assembly 520. It is the cap and valve assembly 524 that connects to a conduit that delivers pumped milk to the container assembly.

Also, as shown in FIGS. 32A-B, a container assembly 530 can also include a flange 532 that is configured to mate with a cover or lid 534 that includes over-molded seals 535 along connecting edges as well as a cap 536 with over molded seals 535. Various configurations of caps are also contemplated. Moreover, in a related approach, beer wax can be used as the material for the cover in this embodiment, or other approaches to container assemblies by cutting or forming the beer wax material into various desired shapes and perimeters, wherein such material can be washed and reused. Alternatively, beer wax material can be employed to form the container in its entirety. In either approach, the edges of the beer wax material is or are pressed together to form a seal to create the container assembly and the same can define a reusable apparatus.

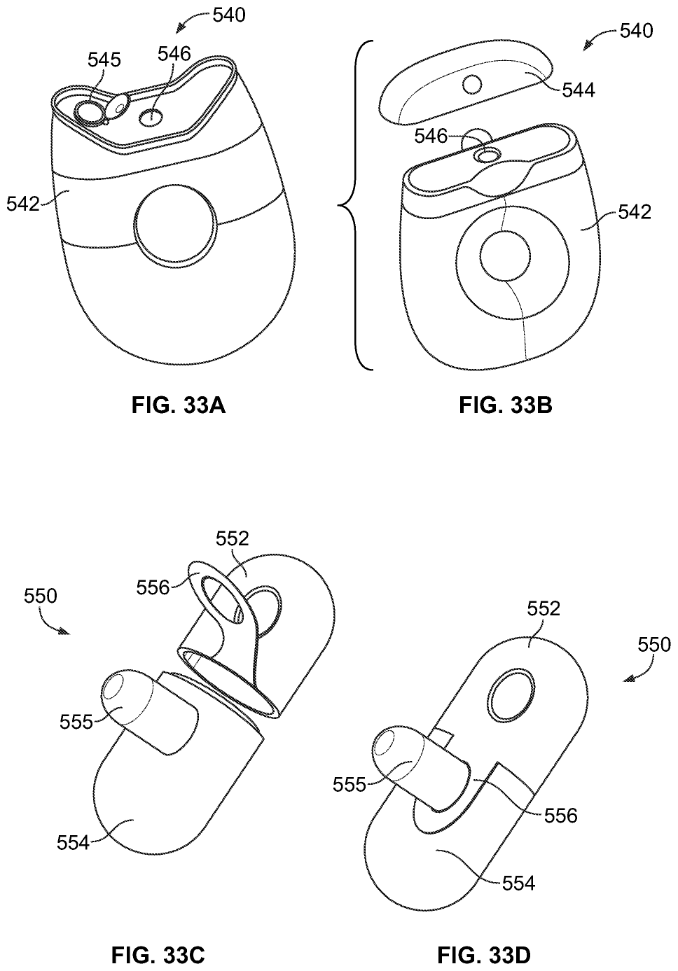

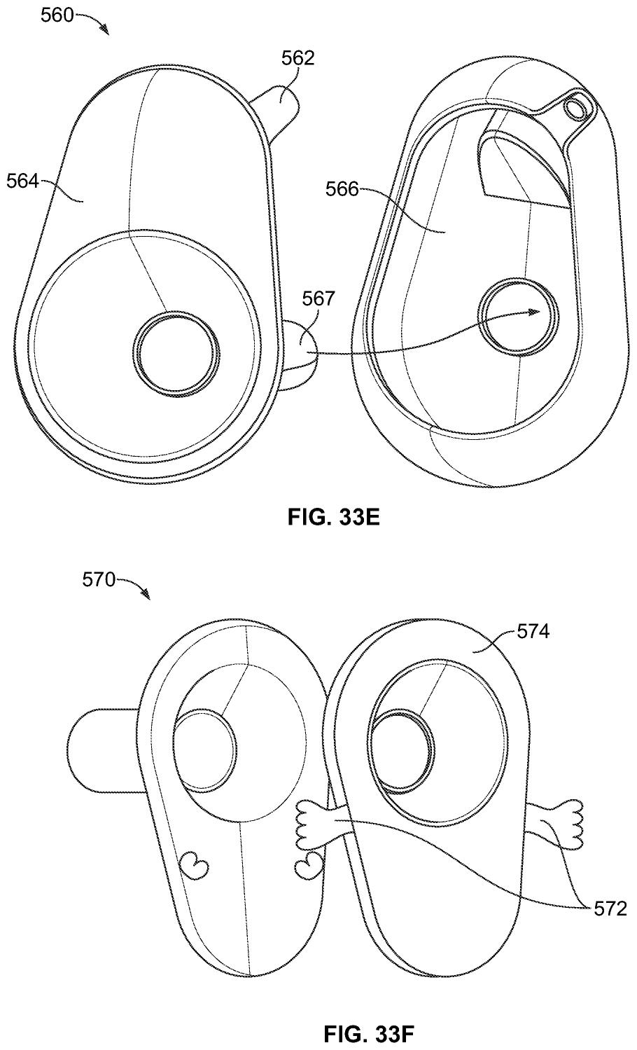

Various additional embodiments of container assemblies are presented in FIGS. 33A-F and include features that can be incorporated into any one of the disclosed approaches to container assemblies. As shown in FIGS. 33A-B for example, a container assembly 540 can include a main body 542 and a cap 544, wherein the main body resembles a canteen with an opening for pouring 545 is configured at the top of the main body 542. The main body 542 also includes breast engaging structure and the nipple receiving portion. The cap 544 completes the assembly and includes structure both intended to mate with a conduit carrying pumped milk as well as a path from such a connection to a fill hole 546 formed in the top of the main body. As with previous embodiments, the completed container assembly completes the breast pump system and accordingly, is configured to releasably mate therewith.

Moreover, as shown in FIG. 33C-D, a container assembly 550 can additionally or alternatively include a top portion 552 and a bottom portion 554. On one side of the container assembly 550, the top and bottom portions cooperate to form a flange for receiving a breast, and on an opposite side, the bottom portion includes structure defining the nipple receiving portion, and an exterior thereof 555. The container assembly 550 further includes a hoop 556 that extends from the top portion 552 and which is sized, shaped and configured to be placed about the exterior of the nipple receiving portion 555 to thus function as an attachment member between the pieces.

Referring now to FIGS. 33E and 33F, there are presented yet further features that can be incorporated into a container assembly. Notably, FIG. 33E shows a container assembly 560 that includes a flap 562 that extends from a perimeter of a first part 564 of a container assembly and which is sized and shaped to fit and attach to a recess formed in a second part of a container assembly 566. In this approach, the first part 564 includes structure functioning as a flange and includes a nipple receiving portion 567. Various sized of this first part 564 are contemplated to be provided to accommodate different breast sizes. The two part container approach 570 approach depicted in FIG. 33F is characterized by including a pair of foldable arms 572 extending from a periphery of a cover 574 component; however, either container assembly part can include such structure.

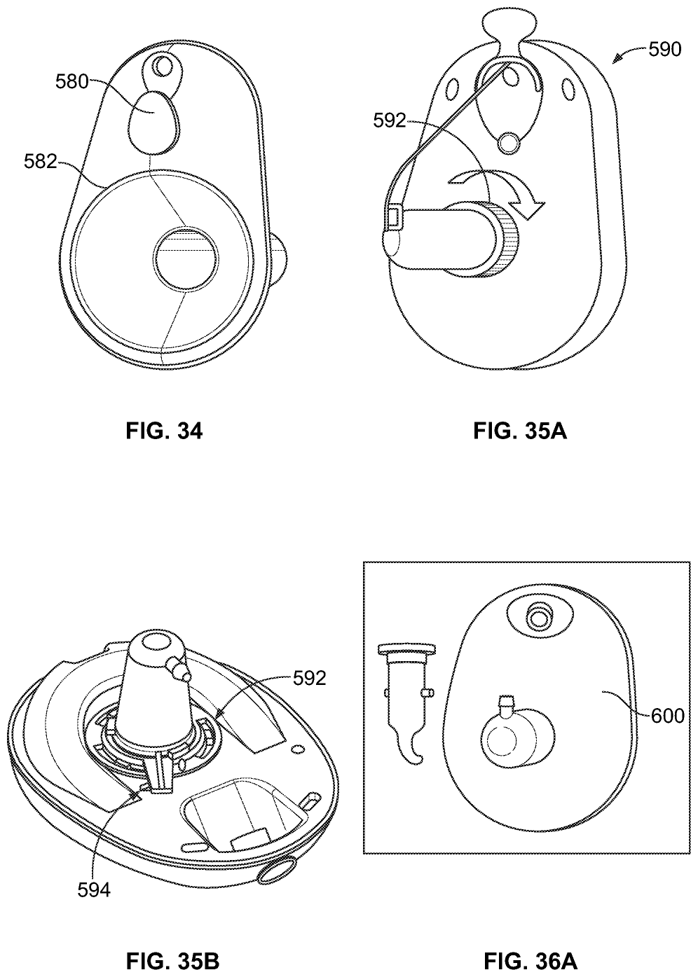

Further, a rotating cover 580 (FIG. 34) can be attached to an external surface of any of the disclosed container assemblies. When in a closed configuration, the cover 580 sits flush with an external surface of the container assembly 582 and seals an entry to within the assembly. A container assembly 590 can further or alternatively include a locking ring 592 (FIGS. 35A-B) that slides over and rotates about an external surface of a nipple receiving portion 594. A half-turn of the locking ring 592 (FIG. 35A), for example, can pull mating pieces of the container assembly 590 together for assembly purposes. In a related approach, the locking ring 592 includes a tab 593 that locks over projections 595 extending from the container assembly to accomplish locking a flange to a container assembly.

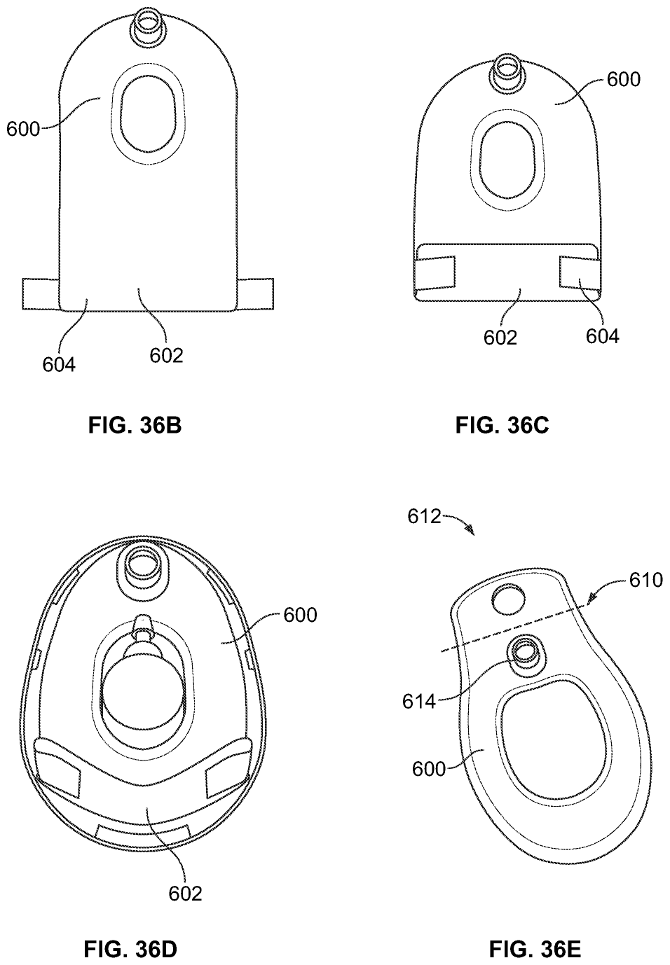

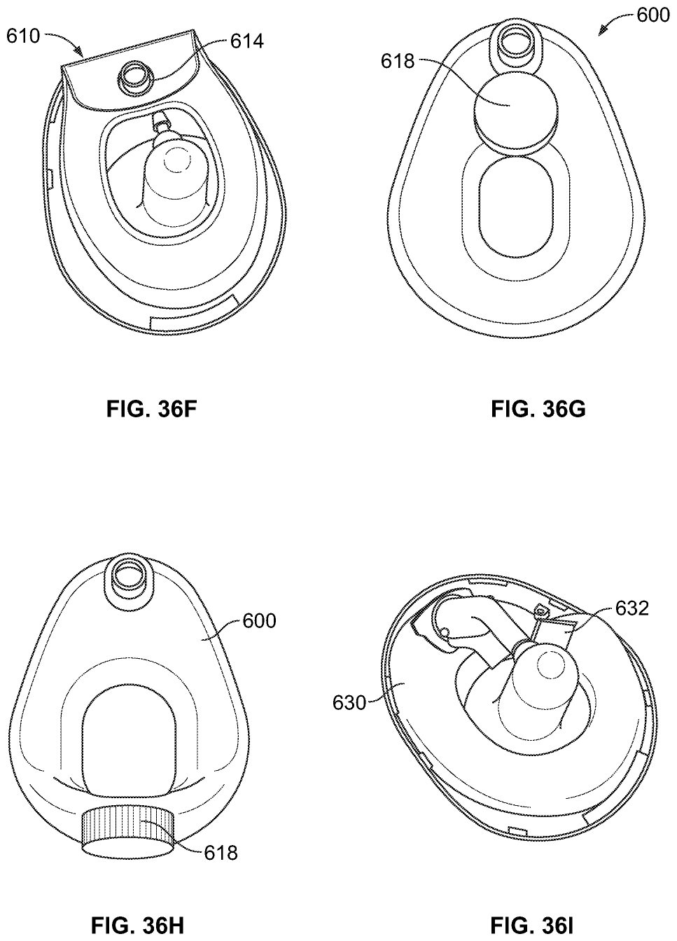

As shown in FIGS. 36A-I, the container assembly can be embodied in a reusable bag container assembly 600 sized and shaped to fit within a breast pump system and to mate with a conduit containing pumped milk. As shown in FIG. 36A, the reusable bag container assembly 600 can be configured to occupy the entirety of an available space within a breast pump system. As shown in FIGS. 36B-D, the container assembly 600 can additionally or alternatively include an open or partially open lower end 602 that is equipped with button, snaps, magnets and/or Velcro 604 for sealing the assembly in a closed configuration. When placed within an interior of a breast pump system, the lower portion 602 of the container assembly is folded upon itself and closed employing the sealing means. In a related approach (FIGS. 36E-F), an upper portion 610 of the container assembly 600 is folded to seal the container 600. As shown, the upper portion 610 includes a hole 612 sized and shaped to fit over a fitment 614 configured within the container. Also, as shown in FIGS. 36G-H, the container assembly 600 can include a removable cap 618 that provides access to an interior of the assembly as well as a convenient port for pouring collected milk. FIG. 361 depicts a bag container assembly 630 that forms a cylinder that wraps about structure internal to the breast pump system. At one end of this assembly is configured a replaceable clamp 632 that seals the structure into a closed configuration.

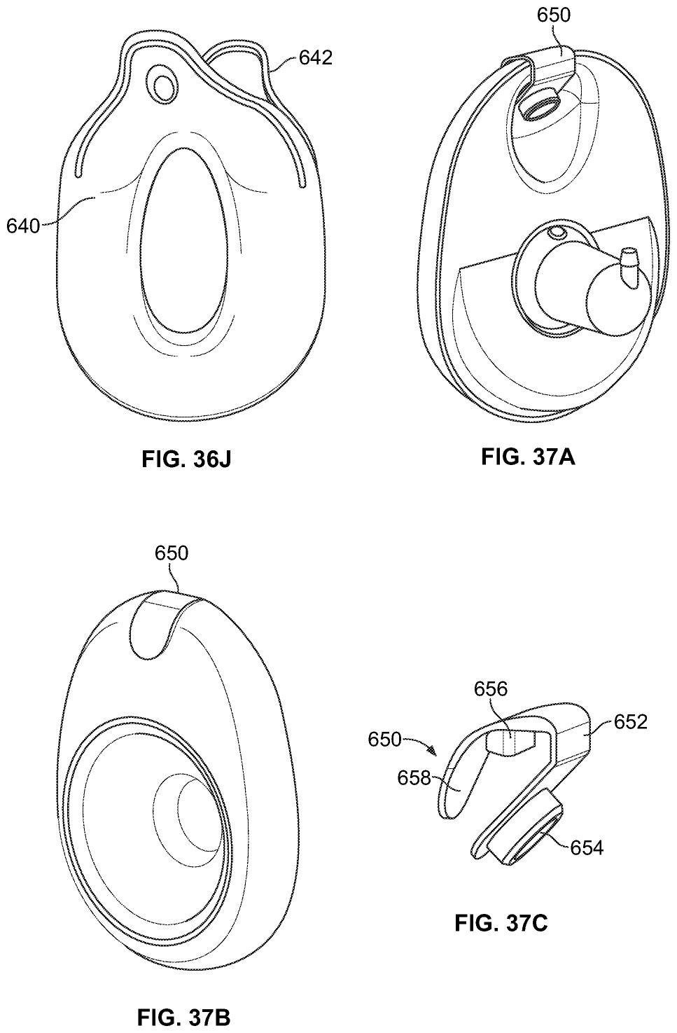

FIG. 361 depicts a reusable bag container assembly 640 that includes a superior edge with zip-lock sealing structure 642. Such structure conveniently seals the assembly 640 for use in a breast pump system and for storing if desired. The contents of the assembly 640 can be removed by separating the zip-lock seal. Moreover, as shown in FIGS. 37A-C, a one-way valve, pour stopper device 650 can be incorporated into one or more of the container assemblies described herein. This device includes a strap 652 connecting a one-way valve portion 654 to a pour spout stopper portion 656. A tab 658 for grasping is also provided at a terminal end of the apparatus. As best seen in FIGS. 37A-B, this device 650 can be attached to a flange or other container assembly and both function as a controlled inlet and a port for pouring collected milk.

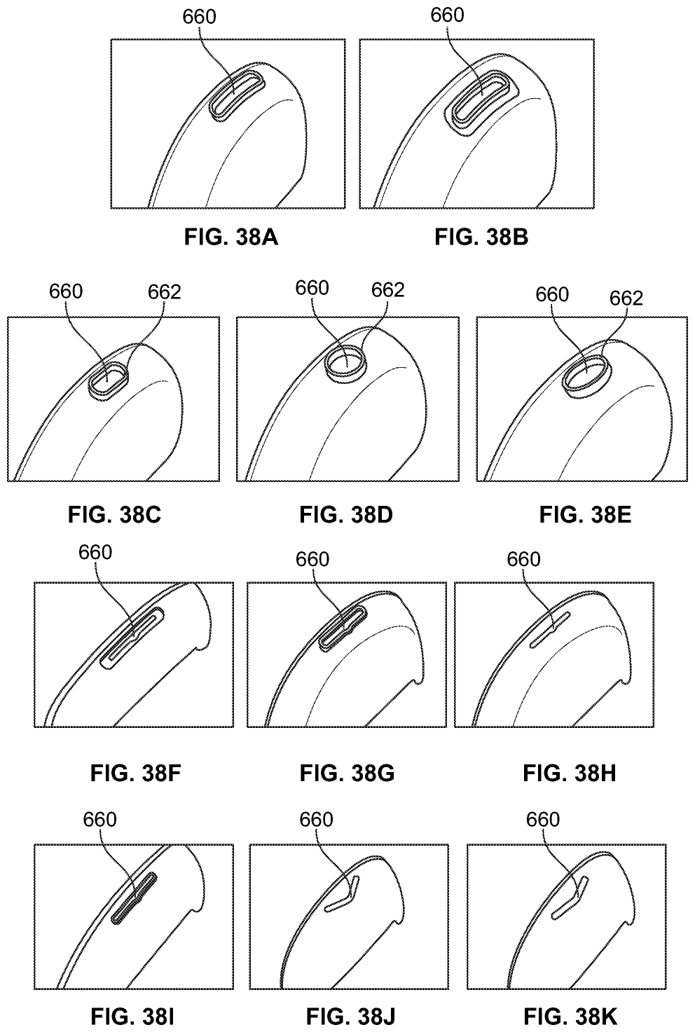

With reference to FIGS. 38A-G, there are shown various alternative approaches to pour spouts 660 for a container assembly. The container assembly can have a cap to seal a pour spout during pumping, transport and storage. The cap can come in various shapes, sizes and are configured to mate with spout located at various locations in the container assembly. Accordingly, the pour spouts 660 may exhibit different geometries and each can accommodate a brush or other cleaning device for easy access cleaning an interior of a container assembly. A lip 662 formed about the spout 660 facilitates controlled milk transfer and minimizes spilling. Moreover, certain spout geometries allow for pouring from multiple orientations, with and without the pump assembly attached. Thus, the pour spout can be biased to one side or centered on a top of a container, for example.

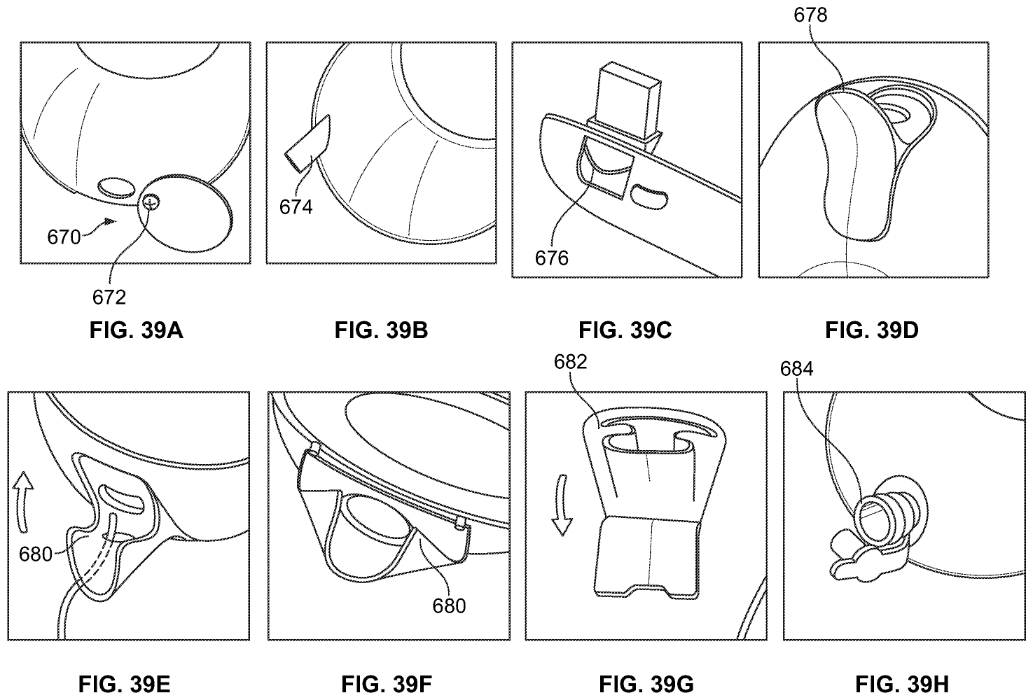

Various other approaches to caps 670 are also included to seal spouts formed in a container assembly and/or various integrated spouts are formed or attached to a container assembly (See FIGS. 39A-G). Caps can be stand-alone, tethered, folded, snapped or over-molded to a container assembly. Further, in addition to sealing a pour spout, a cap may also assist in providing a lip or spout to facilitate milk transfer out of a container. That is, a cap 670 can include one or more of a tether 672 (FIG. 39A), a spout can be embodied in a flip-up straw 674 (FIG. 39B), an internal spout 676 (FIG. 39C), a lipped cap 678 (FIG. 39D), a folding spouts 680 (FIGS. 39E-F), a capped spout 682 (FIG. 39G), or a pull-out spout 684 (FIG. 39H).

Additional approaches to vents or venting a container assembly are presented in

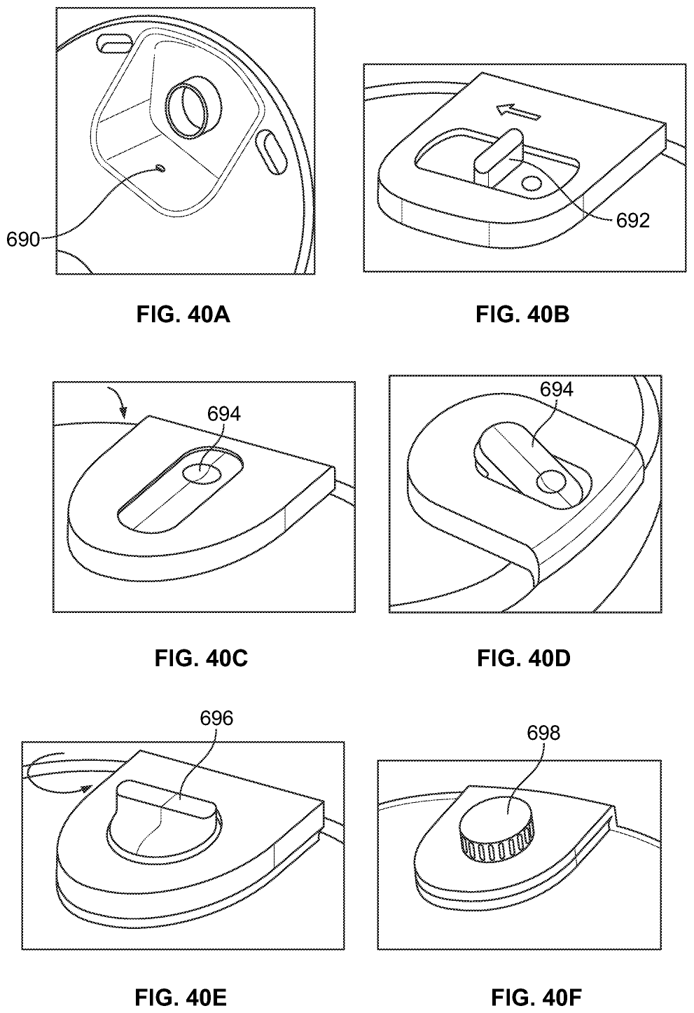

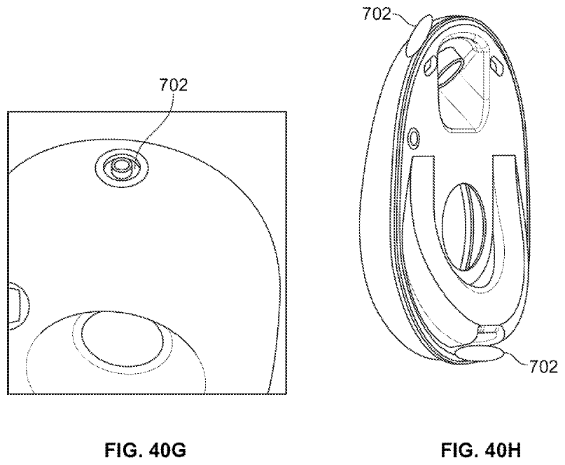

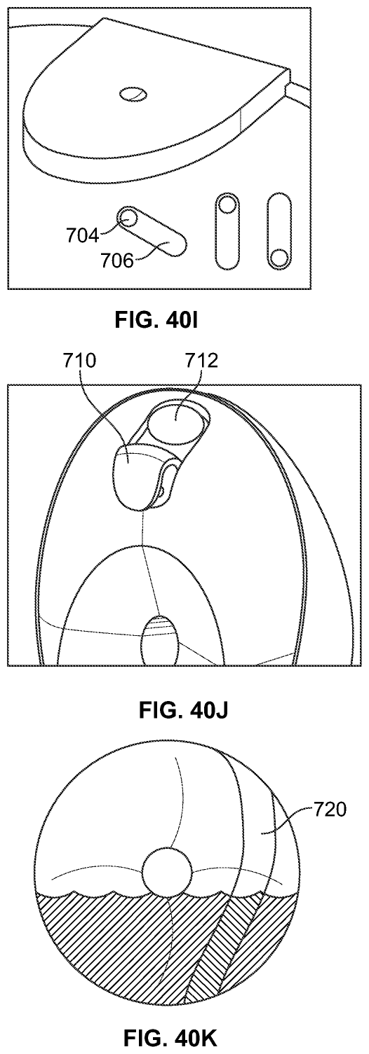

FIGS. 40A-K. In one approach (FIG. 40A), a through-hole 690 is provided in the container assembly to allow the release of air as fluid fills the container. Such a through hole can be the same opening as the pour spout or can define a separate feature. Alternatively, the through hole can be incorporated into a cap for a spout. There can further be structure that functions to open and close the opening to allow the release of air pressure build up. Thus, the closing structure can be embodied in a sliding door feature 692 (FIG. 40B), a pivoting or rotating door 694 (FIGS. 40C-D), a rotating or a twisting cam feature 696 (FIG. 40E), or a twistable cap 698 (FIG. 40F). Alternatively or additionally, the vent structure can include a valve 702 (FIG. 40G), such as an umbrella valve, duckbill valve or diaphragm arrangement, integrated into a container such as in a pour spout or elsewhere to allow air pressure to release as the container fills with fluid. This approach also functions to minimize a potential for fluid to spill as the user moves about. A cap can additionally be provided to seal the valve shut to and define a plug or any of the described structures.

Moreover, as shown in FIG. 40H, two valves 702 integrated into a container, where one valve located at a top of the container would exhibit a lower crack pressure, allowing air to release from it during a pumping session. Here, the user would cap the top valve when completing a pumping session and then perform a flip to finish. The valve at the bottom would have a high crack pressure allowing air release during flipg to finish when the container is inverted. It is to be recognized that in place of valves, porous PTFE membranes can be employed for venting functionality, the membranes allowing air flow but preventing fluid flow. Also, membranes can be hydrophobic and/or oleophobic and membranes would be placed near the top and bottom of the container to allow air to release during standard pumping as well as during flip to finish. Further, hydrophilic PTFE membranes can also be single use where the user applies a PTFE membrane stick over a pour spout at the start of pumping. When pumping is completed, fluid comes in contact with the PTFE membrane and seals the membrane closed to function as a cap until the user is ready to transfer the milk to a separate receptacle.

In one aspect, there can be provided a single valve near a top of a container that remains naturally open and vents air but as the fluid level increases, the valve closes in response to increased pressure. In a single vent approach the container can additionally include an internal diaphragm that expands as pressure builds as additional air and milk is added during final steps in a pumping process such as due to flip to finish, thus eliminating a need for a second valve. As shown in FIG. 401, a container assembly can additionally or alternatively include one or more elastic balls 704 that are configured to ride in a channel 706 to opens to the outside. As fluid enters the container, air is release through the channel. Upon flip to finish, the weight of the fluid pushes against the ball 704 to seal the opening to the channel 706.

As shown in FIG. 40J, a valve can be design into a cap 710 where the cap includes an elastomeric layer that includes a diaphragm or duckbill valve 712. After pumping, the user closes the cap 710 to seal it over the valve 712. As shown in FIG. 40K, a container can additionally or alternatively include an internal channel 720 that functions to evacuate air as the container fills with fluid. The air enters the channel 720 from a top end and exit from the bottom of the container. When the user performs a flip to finish, the container is inverted thus allowing the channel 720 to fill with fluid.

Accordingly, various approaches and features of container assemblies and fitments for a breast pump system have been presented. Each of the disclosed features and structures can be incorporated into a particular approach to a container assembly or breast pump system to define a convenient and effective structure.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the present disclosure.

That which is claimed is:

1. A reusable container assembly for a breast pump system, comprising:
a first part;
a second part sized and shaped to seal with the first part; and
a funnel sized and shaped to receive a breast, the funnel mating with the first and second parts to complete a sealed reusable container assembly.

2. The resuable container assembly of claim 1, wherein the funnel includes a nipple receiving portion.

3. The reusable container assembly of claim 2, wherein the nipple receiving portion includes an outer surface and the first and second parts include a first opening and a second opening, and wherein the outer surface of the funnel mates with and seals against the first and second openings.

4. The reusable container of claim 1, wherein the container assembly is longitudinally split to define the first and second parts.

5. The reusable container of claim 1, wherein the container assembly is laterally split to define the first and second parts.

6. The reusable container of claim 1, wherein the second part includes an oval opening and the flange includes an oval shaped base that mates with the oval opening.

7. The reusable container of claim 1, further comprising a removable plug that defines a connection to a conduit that delivers fluid to the container assembly.

8. The reusable container of claim 1, wherein the first and second parts are welded together.

9. . The reusable container of claim 1, wherein the first and second parts are removably sealed to each other.

10. The reusable container of claim 1, the funnel further including a tab that fits within a recess formed in the first part, the tab and recess facilitating proper positioning of the funnel in the first part.

11. The reusable container of claim 1, wherein the second part includes a scalloped edge that is sized and shaped to mate with a second scalloped edge of the first part.

12. The reusable container of claim 1, further comprising a bi-stable port area including an expanded configuration adapted for pouring.

13. The reusable container of claim 2, further comprising a hoop extending from the second part, the hoop sized and shaped to be placed over the nipple receiving portion.

14. The reusable container of claim 2, further comprising a locking ring sized and shaped to be rotated about the nipple receiving portion.

15. The reusable container of claim 1, further comprising a fitment assembly including one or more components formed from layers of TPE/LDPE/TPE or similar variations so that the TPE parts are utilized as sealing material with mating parts.
